(19) United States
(12) Patent Application Publication
(10) Pub. No.: US 2023/0293229 A1
(43) Pub. Date: Sep. 21, 2023

(54) USING CHARACTERISTICS OF NATIVE OR EVOKED SENSED NEURAL ACTIVITY TO SELECT DENERVATION PARAMETERS

(71) Applicant: Otsuka Medical Devices Co., Ltd., Tokyo (JP)

(72) Inventors: Neil BARMAN, Menlo Park, CA (US); Andrew WU, Los Altos Hills, CA (US); Liang ZHAI, Belmont, CA (US); John W. OSBORN, St. Paul, MN (US); Eric A. Schepis, Alpharetta, GA (US)

(21) Appl. No.: 18/182,821

(22) Filed: Mar. 13, 2023

Related U.S. Application Data
(60) Provisional application No. 63/320,103, filed on Mar. 15, 2022.

(51) Int. Cl. A61B 18/14

(52) U.S. Cl. CPC A61B 18/1492; A61B 2018/00577

(57) ABSTRACT

Tissue treatment systems and methods are disclosed. A tissue treatment system comprises a signal generator, a sensing circuit coupleable to electrode(s) of a catheter, and a controller. The sensing circuit senses neural activity of nerves within tissue surrounding a biological lumen using electrode(s) of the catheter inserted into the biological lumen. The controller determines one or more characteristics of the sensed neural activity of the nerves within the tissue surrounding the biological lumen. The controller also selects one or more denervation parameters based on the one or more characteristics of the sensed neural activity, and controls the signal generator to generate, using the selected one or more denervation parameters, signals for performing a denervation procedure intended to denervate at least some of the nerves for which the neural activity was sensed.

### PRIORITY CLAIM

This application claims priority to U.S. Provisional Pat. Application No. 63/320,103, filed Mar. 15, 2022, titled USING CHARACTERISTICS OF NATIVE OR EVOKED SENSED NEURAL RESPONSE TO SELECT DENERVATION PARAMETERS, which is incorporated herein by reference in its entirety.

### TECHNICAL FIELD

Embodiments of the present technology generally relate to systems, devices and methods for using characteristics of native and/or evoked sensed neural responses sensed from a biological lumen (e.g., a renal artery) to select denervation parameters for use in a denervation procedure, and/or to diagnose a disease state.

### BACKGROUND

The human body’s nervous system includes both the somatic nervous system that provides sense of the environment (vision, skin sensation, etc.) and regulation of the skeletal muscles, and is largely under voluntary control, and the autonomic nervous system, which serves mainly to regulate the activity of the internal organs and adapt them to the body’s current needs, and which is largely not under voluntary control. The autonomic nervous system involves both afferent or sensory nerve fibers that can mechanically and chemically sense the state of an organ, and efferent fibers that convey the central nervous system’s response (sometimes called a reflex arc) to the sensed state information. In some cases, the somatic nervous system is also influenced, such as to cause vomiting or coughing in response to a sensed condition.

Regulation of the human body’s organs can therefore be somewhat characterized and controlled by monitoring and affecting the nerve reflex arc that causes organ activity. For example, the renal nerves leading to a kidney can often cause a greater reflexive reaction than desired, contributing significantly to hypertension. Measurement of the nerve activity near the kidney, and subsequent ablation of renal nerves can therefore be used to control the nervous system’s overstimulation of the kidney, improving operation of the kidney and the body as a whole.

Because proper operation of the nervous system is therefore an important part of proper organ function, it is desired to be able to monitor and change nervous system function in the human body to characterize and correct nervous system regulation of internal human organs.

New medical therapies have been practiced whereby a probe such as a needle, catheter, wire, etc. is inserted into the body to a specified anatomical location and destructive means are conveyed to nerves by means of the probe to irreversibly damage tissue in the nearby regions. The objective is to modulate (e.g., abolish) nerve function in the specified anatomic location. The result is that abnormally functioning physiological processes can be terminated or modulated back into a normal range. Unfortunately, such medical therapies are not always successful because there is no means to assess that the nervous activity has been successfully abolished. An alternative objective can be to increase a physiologic process or modulate it to an abnormal range.

An example is renal nerve ablation to relieve hypertension. Various studies have confirmed the relationship of renal nerve activity with blood pressure regulation. In various renal ablation procedures, a catheter is introduced into a hypertensive patient’s arterial vascular system and advanced into the renal artery. Renal nerves are located in the arterial wall and/or in regions adjacent to the artery. Destructive means are delivered proximate to the renal artery wall to an extent intended to cause destruction of nerve activity. Destructive means include energy such as radio frequency (RF), microwave, cryotherapy, ultrasound, laser or chemical agents. The objective is to reduce or abolish the renal nerve activity. Such nerve activity is an important factor in the creation and/or maintenance of hypertension and abolishment (or reduction) of the nerve activity reduces blood pressure and/or medication burden.

It is known that ablation of the renal nerves, with sufficient energy, is able to effect a reduction in both systolic and diastolic blood pressure. Current methods are said to be, from an engineering perspective, open loop; i.e., the methods used to effect renal denervation do not employ any way of measuring, in an acute clinical setting, the results of applied ablation energies. It is only after application of such energies and a period of time (1-12 months) that the effects of the procedure are known.

The two major components of the autonomic nervous system (ANS) are the sympathetic and the parasympathetic nerves. The standard means for monitoring autonomic nerve activity in situations such as described is to insert very small electrodes into the nerve body or adjacent to it. The nerve activity creates an electrical signal in the electrodes which is communicated to a monitoring means such that a clinician can assess nerve activity. This practice is called microneurography and its practical application is by inserting the electrodes transcutaneously to the desired anatomical location. This is not possible in the case of the ablation of many autonomic nerves proximate to arteries, such as the renal artery, because the arteries and nerves are located within the abdomen and cannot be accessed transcutaneously with any reliability. Thus, the autonomic nerve activity cannot be assessed in a practical or efficacious manner.

### SUMMARY

When a denervation procedure is performed, default denervation parameters are likely to be used during a denervation procedure, which because they are not tailored to a specific patient may lead to an inefficient and/or ineffective procedure. Therefore, it would be beneficial if improved techniques were available to select denervation parameters tailored to a specific patient to improve the efficiency and effectiveness of a denervation procedure, which may be used, for example, to treat hypertension, but is not limited thereto.

Certain embodiments of the present technology relate to tissue treatment systems. In accordance with certain embodiments, a tissue treatment system comprises a signal generator, a sensing circuit coupleable to at least one electrode of a catheter that is insertable into a biological lumen, and a controller communicatively coupled to the signal generator and the sensing circuit. The sensing circuit is configured to sense neural activity of nerves within tissue surrounding the biological lumen using the at least one electrode of the catheter while the catheter is inserted into the biological lumen. The controller comprises one or more processors and is configured to determine one or more characteristics of the sensed neural activity of the nerves within the tissue surrounding the biological lumen, the one or more characteristics indicative of one or more of a size, type, function or health of the nerves and/or indicative of proximity of the nerves relative to the at least one electrode of the catheter. The controller is also configured to select one or more denervation parameters based on the one or more characteristics of the sensed neural activity. Additionally, the controller is configured to control the signal generator to generate, using the selected one or more denervation parameters, signals for performing a denervation procedure intended to denervate at least some of the nerves for which the neural activity was sensed.

Certain embodiments of the present technology relate to tissue treatment methods. In accordance with certain embodiments, a tissue treatment method comprises sensing neural activity of nerves within tissue surrounding a biological lumen, and determining one or more characteristics of the sensed neural activity of the nerves within the tissue surround the biological lumen, wherein the one or more characteristics is/are indicative of one or more of a size, type, function, or health of the nerves, and/or proximity of the nerves relative to at least one electrode of a catheter inserted in the biological lumen. The method also includes selecting one or more denervation parameters based on the one or more characteristics of the sensed neural activity, wherein the one or more denervation parameters is/are for use in performing a denervation procedure intended to denervate at least some of the nerves for which the neural activity was sensed, and wherein the selecting one or more denervation parameters is performed using one or more processors. Additionally, the method includes performing the denervation procedure using the one or more denervation parameters selected to thereby denervate at least some of the nerves for which the neural activity was sensed.

This summary is not intended to be a complete description of the embodiments of the present technology. Other features and advantages of the embodiments of the present technology will appear from the following description in which the preferred embodiments have been set forth in detail, in conjunction with the accompanying drawings and claims.

The details of one or more examples of the invention are set forth in the accompanying drawings and the description below. Other features and advantages will be apparent from the description and drawings, and from the claims.

### BRIEF DESCRIPTION OF THE FIGURES

FIG. 1 is a high level flow diagram used to summarize methods according to certain embodiments of the present technology.

FIG. 2 is a flow diagram that explains a technique for identifying the minimal amount of stimulation energy needed to cause an evoked response, as well as identifying the amount of energy at which saturation of the neural response is achieved, which information are examples of characteristics of sensed neural activity that can be used to select preferred denervation parameters for use in performing a denervation procedure, in accordance with certain embodiments of the present technology.

FIG. 3A shows an example catheter with its two selectively deployable electrodes in their non-deployed positions.

FIG. 3B shows the catheter, which was introduced in FIG. 3A, with its two selectively deployable electrodes in their deployed positions.

FIG. 4 is a schematic diagram of an example system, according to an embodiment of the present technology, for interfacing with a patient’s arterial nerves.

FIGS. 5A and 5B illustrate example cross-sections of a portion of the shaft of the catheter shown in FIGS. 3A and 3B.

FIG. 6 illustrates example details of a fluid supply subsystem introduced in FIG. 4.

FIGS. 7A and 7B illustrates, respectively, a longitudinal cross-sectional view and a radial cross-sectional view of an example transducer of the catheter shown in FIGS. 3A and 3B.

### DETAILED DESCRIPTION

In the following detailed description of example embodiments, reference is made to specific example embodiments by way of drawings and illustrations. These examples are described in sufficient detail to enable those skilled in the art to practice what is described, and serve to illustrate how elements of these examples may be applied to various purposes or embodiments. Other embodiments exist, and logical, mechanical, electrical, and other changes may be made. Features or limitations of various embodiments described herein, however important to the example embodiments in which they are incorporated, do not limit other embodiments, and any reference to the elements, operation, and application of the examples serve only to define these example embodiments. Features or elements shown in various examples described herein can be combined in ways other than shown in the examples, and any such combination is explicitly contemplated to be within the scope of the examples presented here. The following detailed description does not, therefore, limit the scope of what is claimed.

Regulating operation of the nervous system to characterize nerve signaling and modulate organ function includes in some examples introduction of a catheter (aka a probe) into the body to a specified anatomical location (e.g., within a biological lumen, such as a renal artery, but not limited thereto), and at least partially destroying or ablating nerves using the probe to destroy nerve tissue in the region near the probe. By reducing nerve function in the selected location, an abnormally functioning physiological process can often be regulated back into a normal range. It would also be possible to modulate nerve function to purposely cause an abnormally functioning that is beneficial to the patient.

Unfortunately, it is typically difficult to estimate the degree to which nerve activity should be or has been reduced, which makes it difficult to perform a denervation procedure where it is desired to ablate all nerves, or to ablate some, but not all, nerves to bring the nervous system response into a desired range without destroying the nervous system response entirely.

As noted above, when a denervation procedure is performed, preferred denervation parameters (which can also be referred to as ablation parameters) to use for performing the denervation procedure on a specific patient are typically unknown. Rather, default denervation parameters are likely to be used during a denervation procedure, which may lead to an inefficient and/or ineffective procedure.

A denervation procedure may be used, for example, to perform renal nerve ablation (aka renal denervation) to treat hypertension. Various studies have confirmed that renal nerve activity has been associated with hypertension, and that ablation of the renal nerves can improve renal function and reduce hypertension. In a typical procedure, a catheter is introduced into a hypertensive patient’s arterial vascular system and advanced into the renal artery. Renal nerves located in the arterial wall and in regions adjacent to the artery are ablated by destructive means such as RF energy, microwave energy, ultrasound energy, cryotherapy, laser or chemical agents to limit the renal nerve activity, thereby reducing hypertension in the patient.

Unfortunately, renal nerve ablation procedures are sometimes ineffective, such as due to either insufficiently ablating the nerve or destroying more nerve tissue than is desired. Also, it may be desirable to avoid ablating other off-target tissues. Clinicians may estimate, based on provided guideline estimates or past experience, the degree to which application of a particular ablative method will reduce nerve activity, and it can take a significant period of time (e.g., 1-12 months) before the clinical effects of the ablation procedure are fully known.

Some attempt has been made to monitor nerve activity in such procedures using microneurography by inserting very small electrodes into or adjacent to the nerve body, which are then used to electrically monitor the nerve activity. Such microneurography practices are not practical in the case of renal ablation because the renal arteries and renal nerves are located within the abdomen and cannot be readily accessed, making monitoring and characterization of nerve activity in a renal nerve ablation procedure a challenge.

Prior methods such as inserting electrodes into the arteries of a patient’s heart and analyzing received electrical signals are not readily adaptable to renal procedures. In the heart, the ablated tissue is heart muscle which itself is electrically conductive. Further, the cardiac electrical signals emitted from the heart are generally large and slow-moving relative to electrical signals near the renal arteries, which tend to be smaller in size and produce smaller signals that propagate more quickly through the nerves. In the case of renal denervation, the target of the ablation is renal nerves which lie outside the lumen of the blood vessel, and the blood vessel tissue is different from myocardium and acts as a barrier to adequately sense nerve firing. As such, intracardiac techniques used in heart measurements are not readily adaptable to similar renal procedures.

Because nerve activity during procedures to ablate or neuromodulate enervation of organs such as renal nerve ablation cannot be readily measured, it is also difficult to ensure that an ablation probe is located at the most appropriate sites along the renal artery, or to measure the efficiency of the nerve ablation process in a particular patient.

Certain embodiments of the present technology relate to methods, devices and systems that use characteristics of sensed native and/or evoked neural responses to select preferred denervation parameters that can be used to efficiently and effectively perform denervation procedures.

In accordance with certain embodiments of the present technology, a catheter that is inserted into a biological lumen (e.g., a renal artery) is used to sense native (aka spontaneous) neural activity of nerves that surround the biological lumen (e.g., renal nerves that surround a renal artery). Additionally, or alternatively, such a catheter can be used to emit electrical energy (aka stimulation energy) to evoke a neural response that is sensed by the catheter. Either way, characteristics of the sensed neural activity can be quantified and used to tailor patient specific parameters of nerve destruction that is to be performed as part of a medical procedure. This can be the case if one catheter is used for the neural activity sensing and another catheter is used for performing the nerve destruction, or if the same catheter (which can be referred to as an all-in-one catheter) is used for both the neural activity sensing and the nerve destruction (aka denervation). Where electrical energy is emitted (i.e., stimulation is performed) to evoke a neural response, certain additional information may be obtained that would not be obtainable where just native (aka spontaneous) neural activity is sensed, as will be appreciated from the below discussion.

Sensed native neural activity, when plotted in a graph that shows sensed neural energy versus time, typically includes various different peaks of different amplitudes that are temporally spaced apart from one another. The temporal locations or timing of the peaks are indicative of when various nerves fired, and their respective amplitudes may also be indicative of how far the firing nerves are from the sensing electrode(s). Presuming for simplicity that each individual different peak corresponds to a different individual nerve firing, the amplitude of an individual peak may be indicative of an axial radial distance between sense electrode(s) and a nerve, and thus, how deep or shallow the nerve is relative to the lumen wall of the biological lumen within which the catheter is located. Where this is the case, a peak having a relatively large amplitude would be indicative of the nerve being relatively close to the lumen wall (i.e., shallow), and a peak having a relatively small amplitude would be indicative of the nerve being relatively far from the lumen wall (i.e., deep). The amplitude of an individual peak may alternatively or additionally be indicative of a longitudinal distance between the sense electrodes and the nerve, and thus, how far along the blood vessel (or other type of biological lumen) the nerve is located relative to the sense electrode(s). Where this is the case, a peak having a relatively large amplitude would be indicative of the nerve being longitudinally close to the sense electrode(s), and a peak having a relatively small amplitude would be indicative of the nerve being longitudinally far from the sense electrode(s). The amplitude of an individual peak may alternatively or additionally be indicative of a size of a nerve, with a larger nerve producing a larger peak amplitude than a smaller nerve. It is also possible that different sensed amplitudes are at least in part due to one type of nerve (e.g., an efferent nerve) firing more strongly than another type of nerve (e.g., an afferent nerve). The peaks of a sensed signal indicative of neural activity can also be indicative of the function and/or health of the nerves for which the neural activity is sensed.

Regardless of the exact reason for the various peak amplitudes that may be sensed, the magnitudes of such peak amplitudes can be used to tailor parameters of nerve destruction that is to be performed as part of a medical procedure. For example, if the nerve destruction process is being performed using RF energy, microwave energy, or ultrasound energy, which can be collectively referred to as ablation energy, then the amplitudes of the peaks included in a sensed neural activity signal can be used to select specific parameters of the ablation energy, such as, but not limited to, the amplitude, power, duration, near-field cooling, frequency, and/or duty cycle of the ablation energy. In certain embodiments, the greater the magnitude of one or more of the above mentioned characteristics, the greater the parameters selected to perform a denervation parameter.

Where there are multiple peaks in a sensed signal indicative of neural activity, an average and/or median amplitude can be determined, and that average and/or median amplitude can be what is used to select one or more parameters of the ablation energy that is to be delivered. Alternatively or additionally, a curve can be fit to a portion of the sensed signal indicative of neural activity, and an area under the curve can be what is used to select one or more parameters of the ablation energy. In certain embodiments, the greater the magnitude of one or more of the above mentioned characteristics, the greater the parameters selected to perform a denervation parameter.

Alternatively or additionally, the number of different peaks could be categorized into different groupings such that the quantity within each grouping or relative ratios between the groupings could be used to select one or more parameters of the ablation energy. In accordance with certain embodiments, different peaks can be categorized into different groups based on amplitudes, with smaller amplitude peaks (e.g., peaks below a specified amplitude) representing one or more of the following: smaller size nerves, nerves of a specific subtype (e.g., afferent nerves), or nerves at a greater distance from the biological lumen; and with larger amplitude peaks (e.g., peaks above a specified threshold) representing one or more of the following: larger size nerves, nerves of another specific subtype (e.g., efferent nerves), or nerves at a closer distance from the biological lumen. If there are more smaller amplitude peaks than larger amplitude peaks, that can be interpreted as there being some distant nerves that need to be denervated. Accordingly, in response thereto, denervation parameters can be selected to achieve deeper lesions, e.g., by selecting relative large amplitude and/or long duration parameters. If there are more larger amplitude peaks than smaller amplitude peaks, that can be interpreted as there being closer and/or larger nerves that need to be denervated. Accordingly, in response thereto, denervation parameters can be selected to achieve shallower lesions, e.g., by selecting relatively low amplitude and/or short duration parameters. If different peaks are categorized into different groups following a denervation procedure having already been performed, than the number of smaller amplitude peaks relative to the number of larger amplitude peaks can be used to quantify the efficacy of the denervation procedure that had been performed, as well as to determine whether an additional denervation procedure should be performed, and if so, which parameters to select.

The aforementioned measurements can be used to select initial ablation energy parameters. More specifically, prior to any ablation being performed, a catheter can be used to sense native neural activity and/or evoked neural activity, wherein the neural activity that is sensed prior to the any ablation being performed can be referred to as baseline neural activity. Accordingly, the sensed baseline neural activity can be native neural activity and/or evoked neural activity. Characteristics of the sensed baseline neural activity can be used to select initial parameters of the ablation energy. After the initial ablation is performed, a catheter can again be used to sense native neural activity and/or evoked neural activity, and the post-ablation neural activity can be compared to the pre-ablation baseline neural activity. Results of this comparison can then be used to determine whether and/or to what extent further ablation energy should be delivered. If there is no or minimal post-ablation neural activity, then it may be determined that sufficient nerve destruction has occurred, and that no further ablation is needed. However, if there is still more post-ablation neural activity than desired (e.g., if neural activity exceeds a respective specified threshold), then further ablation energy can be delivered to perform further nerve destruction.

In accordance with certain embodiments, one or more parameters of the further ablation energy that is to be delivered is/are selected based on the post-ablation neural activity that is sensed using a catheter. In some embodiments, these one or more parameters are selected based on the sensed post-ablation neural activity, without regard to how the sensed post-ablation neural activity compares to the sensed pre-ablation neural activity. Alternatively, the sensed post-ablation neural activity can be compared to the sensed pre-ablation neural activity, and results of the comparison can be used to select one or more parameters of the further ablation energy. For an example, if the sensed pre-ablation neural energy activity including numerous high amplitude peaks, and the sensed post-ablation neural activity energy including primarily only low amplitude peaks, then it may be determined that the further ablation energy can have a lower magnitude and/or be delivered for a shorter amount of time than the initial ablation energy. For another example, if the sensed pre-ablation neural activity energy includes numerous high amplitude peaks and numerous low amplitude peaks, and the sensed post-ablation neural energy includes primarily only high amplitude peaks, then it may be determined that the further ablation energy should have a higher magnitude and/or be delivered for a longer amount of time (i.e., a longer duration) than the initial ablation energy.

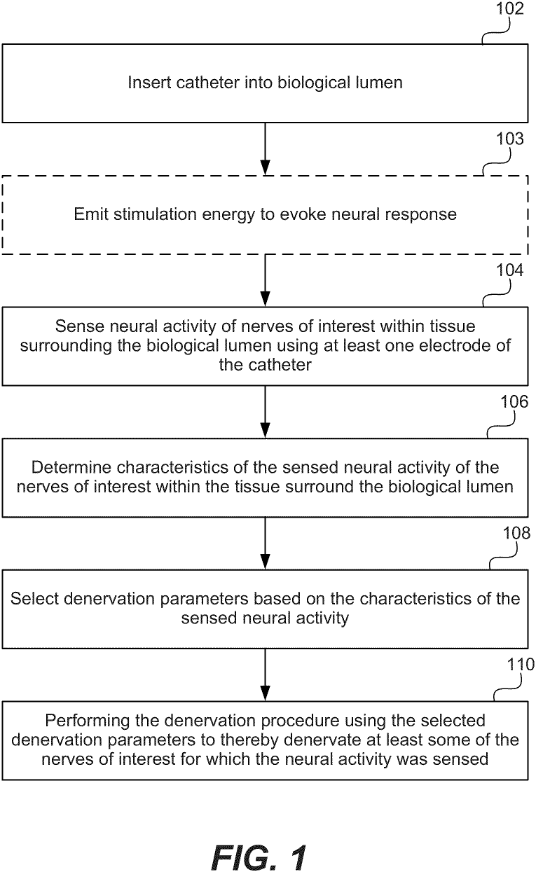

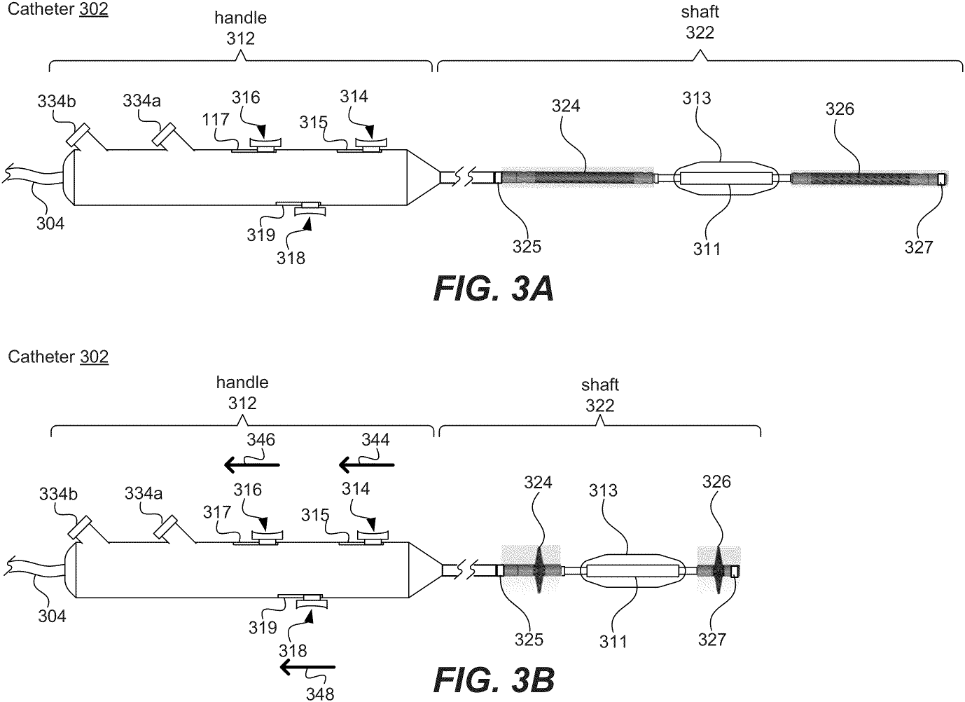

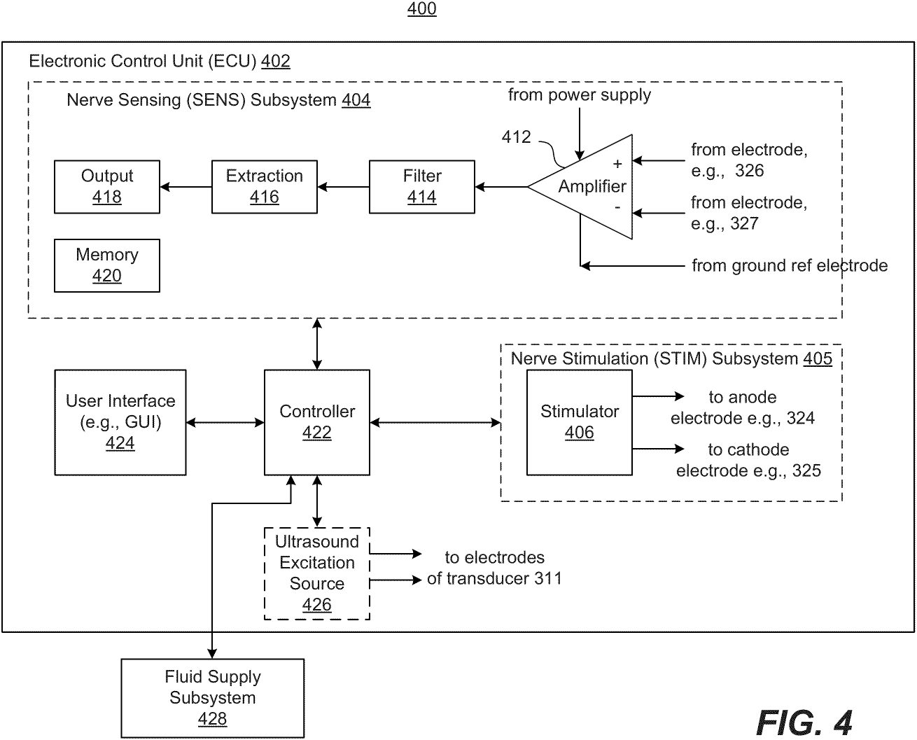

The high level flow diagram of FIG. 1 will initially be used to summarize methods according to certain embodiments of the present technology. Referring to FIG. 1, (as well as FIGS. 3A, 3B and 4) step 102 involves inserting a catheter (e.g., 302) into a biological lumen (e.g., a renal artery) so that a distal portion of the catheter (e.g., 302), which includes one or more electrodes (e.g., 324, 325, 326, 327), is positioned at a desired location with the biological lumen. Step 104 involves sensing neural activity of nerves within tissue surrounding a biological lumen (e.g., using a sensing circuit 404 couplable to the one or more electrodes 324, 325, 326, 327 of the catheter 302, wherein the sensing can be performed using at least one electrode of the catheter inserted into the biological lumen). Electrodes (e.g., 326, 327) that are used to sense neural activity can be referred to herein as sensing electrodes. The neural activity that is sensed at step 104 (e.g., by the sensing circuit 404) can be native (aka spontaneous) neural activity. Alternatively, or additionally, the neural activity that is sensed (e.g., by the sensing circuit 404) at step 104 can be evoked neural activity that is evoked by electrical stimulation delivered (e.g., using the using the same catheter used to sense the neural activity at step 104. More specifically, in certain embodiments, between steps 102 and 104 one or more electrodes of the catheter is/are used to emit stimulation energy (e.g., output by the stimulation circuit 405) at step 103 (shown in a dashed line block) that causes an evoked neural response that is sensed (e.g., by the sensing circuit 404) at step 104. Electrodes (e.g., 324, 325) that are used to evoke neural activity can be referred to herein as stimulation electrodes. An example of a catheter 302 that includes electrodes 324, 325, 326, 327 and can be used to perform steps 102 and 104, as well as to selectively emit stimulation energy to evoke a neural response at step 103, is described below with reference to FIGS. 3A and 3B. However, it should be noted that embodiments of the present technology are not limited to being used with the example catheter described below with reference to FIGS. 3A and 3B.

Still referring to FIG. 1, step 106 involves determining one or more characteristics of the sensed neural activity of the nerves within the tissue surround the biological lumen, wherein the characteristic(s) is/are indicative of one or more of the size, type, function or health of the nerves for which neural activity is sensed and/or indicative of proximity of the nerves relative to the sensing electrode(s) of the catheter, but not limited thereto. For example, step 106 can be performed by a controller 422 of the system 400 described below with reference to FIG. 4.

Step 108 involves selecting one or more denervation parameters based on the characteristic(s) of the sensed neural activity, wherein the denervation parameter(s) is/are for use in performing a denervation procedure intended to denervate at least some of the nerves for which the neural activity was sensed. For example, step 108 can be performed by the controller 422 of the system 400 described below with reference to FIG. 4. In accordance with certain embodiments, the selecting of denervation parameter(s) at step 108 is performed using one or more processors, e.g., of an electrical control unit (ECU), an example of which is described below with reference to FIG. 4. Such an ECU can include a controller (e.g., 422 in FIG. 4), wherein the controller includes one or more processors. In certain embodiments, one or more denervation parameters is/are selected using one or more tables stored in memory (e.g., 420 in FIG. 4) of an ECU (e.g., 402 in FIG. 4) and accessed by at least one processor (e.g., of a controller 422 in FIG. 4). In other embodiments, one or more denervation parameters is/are selected using a machine learning model implemented by at least one of the one or more processors, or more generally, using artificial intelligence.

Step 110 involves performing the denervation procedure using the selected denervation parameter(s) to thereby denervate at least some of the nerves for which the neural activity was sensed. This step 110 can involve the controller 422 controlling a signal generator (e.g., 406) to generate signals for performing the denervation procedure using the selected one or more denervation parameters. Depending upon the specific implementation, the denervation procedure can be performed at step 110 using the same catheter (e.g., 302) used to sense the neural activity at step 104, or using a separate catheter inserted into the biological lumen (e.g., a renal artery) after the catheter used to sense the neural activity at step 104 has been removed. In other words, in certain embodiments, one catheter may be swapped for another catheter during a time period between when step 104 is performed and when step 110 is performed. Alternatively, an all-in-one catheter (e.g., 302) can be used to perform both of steps 104 and 110, in which case there is no need to swap catheters during the time period between when step 104 is performed and when step 110 is performed.

In accordance with certain embodiments, the biological lumen referred to in the flow diagram of FIG. 1 is a renal artery and the nerves comprise renal nerves innervating a kidney. The method can also include removing any catheter (e.g., 302) that had been inserted into a biological lumen, such as a renal artery. Where a denervation procedure described herein is being performed using a catheter (e.g., 302) inserted into a renal artery type of biological lumen, the disease being treated using the denervation procedure can be hypertension or some other disorder associated with elevated sympathetic nerve activity, as can be appreciated from the above discussion. However, it is noted that embodiments of the present technology described herein can alternatively be used to improve the performance of denervation procedures using catheters that are inserted into other types of biological lumens, besides a renal artery, to treat other types of diseases besides hypertension. For example, such other types of biological lumens include a vein, a pulmonary artery, a vascular lumen, a celiac artery, a common hepatic artery, a proper hepatic artery, a gastroduodenal artery, a hepatic artery, a splenic artery, a gastric artery, a blood vessel, a nonvascular lumen, an airway, a sinus, an esophagus, a respiratory lumen, a digestive lumen, a stomach, a duodenum, a jejunum, a cancer tissue, a tumor, an intestine, and a urological lumen, but are not limited thereto. Examples of other types of diseases that can be treated using an embodiment of the present technology include pulmonary hypertension, diabetes, obesity, nonalcoholic fatty liver disease, heart failure, end-stage renal disease, digestive disease, cancers, tumors, pain, asthma or chronic obstructive pulmonary disease (COPD), but are not limited thereto.

In accordance with certain embodiments, the denervation procedure that is performed at step 110 uses an ultrasound transducer (e.g., 311) of a catheter (e.g., 302) inserted into the biological lumen to emit ultrasound energy, which can also be referred to as ultrasonic energy. In certain such embodiments, the denervation parameter(s) selected at step 108 (e.g., by the controller 422), and used at step 110, can specify an amplitude, power, duration, frequency, and/or duty cycle of the ultrasound energy. In certain embodiments, the ultrasound transducer (e.g., 311) is located within a balloon (e.g., 311) that is at least partially filled with a cooling fluid (e.g., 613) that is circulated through the balloon in order to cool at least a portion of the tissue surrounding the biological lumen proximate the balloon, e.g., as described below with reference to FIGS. 3A through 6. In certain such embodiments, the denervation parameter(s) selected at step 108 (e.g., by the controller 422), and used at step 110, can specify a flow rate and/or a temperature associated with the cooling fluid.

In accordance with certain embodiments, the denervation procedure that is performed at step 110 uses one or more electrodes of a catheter that is inserted into the biological lumen to emit RF energy. In certain such embodiments, the denervation parameter(s) selected at step 108, and used at step 110, can specify an amplitude, power, duration, frequency, and/or duty cycle of the RF energy. The use of other mechanisms to perform a denervation procedure, such as microwave energy, chemical, cryotherapy, laser, or pulsed electric field, but not limited thereto, are also within the scope of the embodiments described herein. Denervation parameter(s) for use with such other types of denervation procedures can be selected at step 108 (e.g., by the controller 422) and used at step 110.

Step 104 in FIG. 1 can be performed by using a pair of electrodes (e.g., 326, 327), at least one of which is on the catheter (e.g., 302) inserted into the biological lumen at step 102, to sense a signal indicative of the neural activity, wherein the signal indicative of the neural activity including multiple peaks temporally spaced apart from one another. The electrodes (e.g., 326, 327) that are used for sensing neural activity (whether spontaneous or evoked) can be referred to herein as sensing electrodes, and electrodes (e.g., 324, 325) that are used to deliver stimulation energy to evoke a neural response can be referred to herein as stimulation electrodes, as was noted above. It is noted that switches within a catheter and/or within an electronic control unit (ECU) (e.g., 402) to which the catheter (e.g., 302) is electrically connected can be used to selectively change certain electrodes from sensing electrodes to stimulation electrodes, and/or vice versa, at different points in time. In accordance with certain embodiments, both electrodes of a pair of sensing electrodes are located on the catheter (e.g., 302). In other embodiments, one sensing electrode of a pair of sensing electrodes is located on the catheter (e.g., 302), while another sensing electrode of the pair is located in the distal end of an introducer sheath (that is used to insert the catheter, e.g., 302, into the biological lumen), or on the distal end of a guide wire (that is used to guide the catheter, e.g., 302, into the biological lumen), or alternatively is an external skin electrode located on the skin of a patient.

In certain embodiments, characteristic(s) of the sensed neural activity is/are determined at step 106 (e.g., by the controller 422) based on amplitudes of the multiple peaks and/or based on temporal spacings between the multiple peaks of the sensed signal indicative of the neural activity. For example, an average amplitude of the multiple peaks can be determined and one or more denervation parameters can be selected (e.g., by the controller 422) based on the average amplitude of the multiple peaks. Where the average amplitude of the multiple peaks is relatively large, e.g., above a specified threshold (e.g., of 2.0 µV), that means there is relatively high neural activity that is sensed and that a relatively high amplitude and/or relatively long duration denervation therapy should be selected at step 108 and used at step 110. Where the average amplitude of the multiple peaks is relatively small, e.g., below a specified threshold (e.g., of 2.0 µV), that means there is relatively low neural activity that is sensed and that a relatively low amplitude and/or relatively short duration denervation therapy should be selected (e.g., by the controller 422) at step 108 and used at step 110. For a more specific example, once the average amplitude of the multiple peaks is below a specified threshold (e.g., of 2.0 µV), the amplitude and/or duration of the denervation therapy can be decreased by 50% compared to when the median amplitude of the multiple peaks is above the specified threshold.

Alternatively, or additionally, a median amplitude of the multiple peaks can be determined and denervation parameters can be selected based on the median amplitude of the multiple peaks. For example, a median amplitude of the multiple peaks can be determined and one or more denervation parameters can be selected (e.g., by the controller 422) based on the average amplitude of the multiple peaks. Where the median amplitude of the multiple peaks is relatively large, e.g., above a specified threshold (e.g., of 2.0 µV), that means there is relatively high neural activity that is sensed and that a relatively high amplitude and/or relatively long duration denervation therapy should be selected at step 108 and used at step 110. Where the median amplitude of the multiple peaks is relatively small, e.g., below a specified threshold (e.g., of 2.0 µV), that means there is relatively low neural activity that is sensed and that a relatively low amplitude and/or relatively short duration denervation therapy should be selected (e.g., by the controller 422) at step 108 and used at step 110. For a more specific example, once the median amplitude of the multiple peaks is below a specified threshold (e.g., of 2.0 µV), the amplitude and/or duration of the denervation therapy can be decreased by 50% compared to when the median amplitude of the multiple peaks is above the specified threshold.

Alternatively, or additionally, determining one or more characteristics of the sensed neural activity at step 106 (e.g., by the controller 422) can involve fitting a curve to a portion of the signal indicative of the neural activity, and determining an area under the curve, and step 108 can involve the selecting denervation parameters based on the area under the curve. Where the area under the curve is relatively large, e.g., above a specified threshold, that means there is relatively high neural activity that is sensed and that a relatively high amplitude and/or relatively long duration denervation therapy should be selected at step 108 (e.g., by the controller 422) and used at step 110. Where the area under the curve is relatively small, e.g., below a specified threshold, that means there is relatively low neural activity that is sensed and that a relatively low amplitude and/or relatively short duration denervation therapy should be selected at step 108 (e.g., by the controller 422) and used at step 110. For a more specific example, once the area under the curve is below a specified threshold, the amplitude and/or duration of the denervation therapy can be decreased by 50% compared to when the area under the curve is above the specified threshold.

In each of the above embodiments, the denervation procedure can be considered complete when the average, median, or area under the curve is below a corresponding specified threshold. For example, in certain embodiments, where the average or median amplitude of multiple peaks is below 0.5 µV, then it can be concluded the neural activity is sufficient low such that no (or no further) denervation therapy is needed.

Each nerve that surrounds a biological lumen (for which neural activity is being sensed) will typically fire only once per cardiac cycle. Accordingly, where the temporal spacings between sensed peaks is small relative to a cardiac cycle length, that can be interpreted as there being many nerves firing during the cardiac cycle. By contrast, where the temporal spacing between sensed peak is large relative to a cardiac cycle length, that can be interpreted as there being relatively few nerves firing during the cardiac cycle. Such observations can be used to determine whether a denervation procedure, or a further denervation procedure, should be performed, and/or to select one or more denervation parameters.

As noted above, in certain embodiments stimulation is emitted (at step 103) in order to evoke a neural response to the simulation. This can be achieved be emitting the stimulation using a first electrode (e.g., of a first pair of electrodes 324, 325) on the shaft (e.g., 322) of a catheter (e.g., 302) and sensing the evoked response at a second electrode (e.g., of a second pair of electrodes 326, 327) of the catheter (e.g., 302) that is a known distance from the first electrode. The electrodes (e.g., 324, 325) that are used for stimulating nerves to evoke a neural response can be referred to herein as stimulation electrodes, as was noted above. As was also noted above, switches within a catheter (e.g., 302) and/or within an electronic control unit (ECU) (e.g., 402) to which the catheter is electrically connected can be used to selectively change certain electrodes from sensing electrodes to stimulation electrodes, and/or vice versa, at different points in time. In accordance with certain embodiments, both electrodes of a pair of stimulation electrodes (e.g., 324, 325) are located on the catheter (e.g., 302). In other embodiments, one stimulation electrode of a pair of stimulation electrodes (e.g., 324, 325) is located on the catheter (e.g., 302), while another stimulation electrode of the pair is located in the distal end of an introducer sheath (that is used to insert the catheter into the biological lumen), or on the distal end of a guide wire (that is used to guide the catheter into the biological lumen), or alternatively is an external skin electrode located on the skin of a patient.

In accordance with certain embodiments, there is a determination of a delay (aka latency) between when the stimulation is delivered and when an evoked neural response (to the stimulation) is detected, which delay (aka latency) is indicative of a conduction velocity of the nerves that responded to the stimulation. Different nerve fibers can have different conduction velocities. Some electrical impulses travel faster than others. Larger neural fibers tend to have very fast conduction velocities. By contrast, smaller neural fibers tend to have relatively slower conduction velocities compared to the larger neural fibers. Accordingly, the latency of the sensed electrical impulses can be used to determine the type, size, function and/or health of the fibers whose neural response is being sensed. The delay (aka latency) may be indicative of a depth of nerves surrounding a biological lumen (e.g., a renal artery) within which a catheter used to measure the delay is located. In accordance with certain embodiments of the present technology, the above described delay (aka latency) is another example of a characteristic of the sensed neural activity that can be determined (e.g., by the controller 422) at an instance of step 106 in FIG. 1, and which can be used to select one or more denervation parameters at an instance of step 108 in FIG. 1.

When a nerve fiber is being ablated during a denervation procedure, such that the health of the nerve fibers has been diminished or the nerve has been destroyed, the amplitude and shape of a sensed signal indicative of the neural activity, its latency, and its synchrony will change, compared to such characteristics prior to the denervation procedure. In certain embodiments, one or more of these various characteristics, such as amplitude, shape, latency, and synchrony, are used to quantify a neural fiber’s health.

In accordance with certain embodiments, convolution is used to determine how far nerve fibers are from a sensing electrode (e.g., 326, 327), or more generally from a sensing site (which can also be referred to as a recording site). Typically, the larger the amplitude of the sensed neural activity the more likely nerve fibers are close to the sensing site, and the smaller the amplitude of the sensed neural activity the more likely the nerve fibers are far from the sensing site. Assume for example the neural activity of two different nerve fibers are sensed using a sensing electrode on a catheter, and that one of the nerve fibers is relatively far away from the sensing site, while another one of the nerve fibers is relatively close to the sensing site. Also assume that both the relatively far and the relatively close nerve fibers fire at the same time in response to stimulation energy that is intended to evoke a neural response. When a catheter is used to sense the above described neural activity, characteristics of both the relatively far and the relatively near neural fibers can be distinguished from one another in a sensed signal, which can enable a system and/or physician to approximate how far the different neural fibers are from the sensing site, as well as the size of such fibers, which are examples of characteristics of sensed neural activity that can be determined (e.g., by the controller 422) at step 106 in FIG. 1.

In certain embodiments, sensed neural activity following a denervation procedure is compared to baseline neural activity sensed prior to the denervation procedure to determine the efficacy of the denervation procedure. Based on such a comparison, there can be a determination of whether the denervation procedure was sufficiently successful such that it could be terminated, or whether further ablation energy should be applied because sufficient nerve destruction has not yet been achieved. After enough data has been collected from a patient population, it may also be possible to analyze neural activity from a patient without requiring any baseline measurements. However, until such sufficient patient population data is collected, determining baseline measurements prior to a procedure, and comparing those to post-procedure measurements, is likely a good way to quantify the efficacy of the denervation procedure and to determine whether additional denervation treatment is needed.

In certain embodiments, neural activity can be sensed (e.g., by the sensing circuit 404) and used (e.g., by the controller 422) to diagnose a disease state of a patient, such as to diagnose a patient as having hypertension, since a patient with hypertension will have a neural activity signature indicative of hypertension that is distinguishable from the neural activity signature of a healthy patient that is not experiencing hypertension.

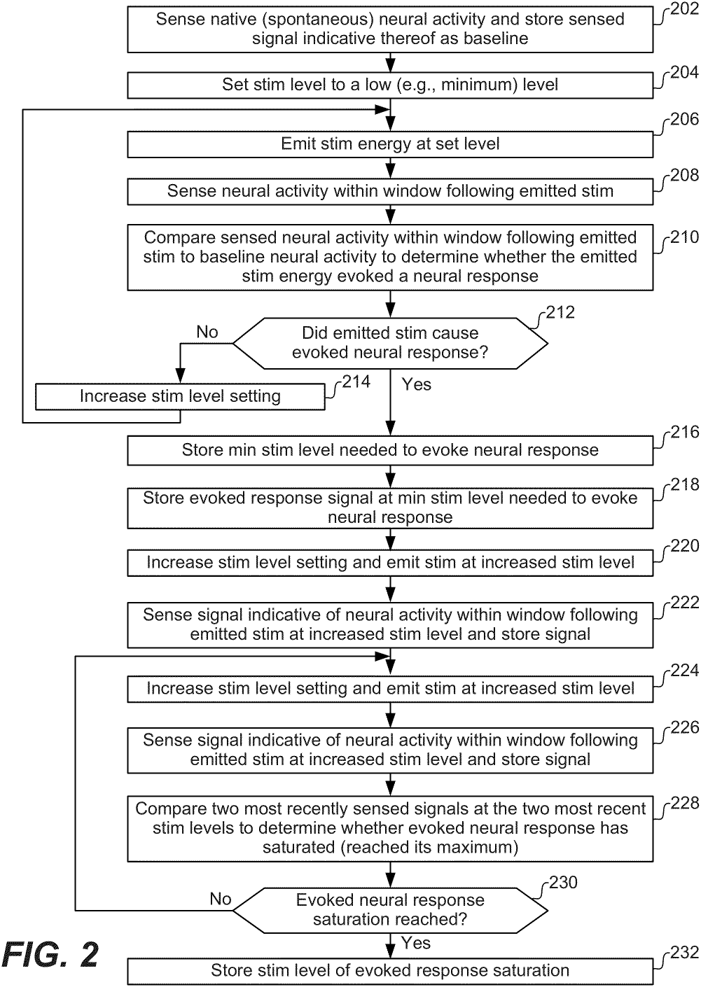

In accordance with certain embodiments, the characteristics of the sensed neural activity that are identified (e.g., by the controller 422) at step 106, and used (e.g., by the controller 422) to select denervation parameters at step 108, include a minimum amount of energy delivered with a specific waveform to get an evoked response and/or the amount of energy delivered with the specific waveform that achieves saturation of the evoked neural response. The evoked neural response can be considered saturated when increases to the neural stimulation no longer increase the evoked neural response. These characteristics can be used to select an amount of therapy to be delivered to the nerve fiber to get full nerve capture and a full effect. More generally, these characteristics can be used (e.g., by the controller 422) to select denervation parameters at step 108 in FIG. 1. The flow diagram of FIG. 2 explains one technique (e.g., used by the system 400) for identifying the minimal amount of stimulation energy needed to cause an evoked response, as well as for identifying the amount of energy at which saturation of the neural response is achieved.

Referring to FIG. 2, step 202 involves sensing (e.g., by the sensing circuit 404) native (i.e., spontaneous) neural activity using one or more sensing electrodes (e.g., 326, 327) of a catheter (e.g., 302) that is inserted into a biological lumen (e.g., a renal artery), and storing (e.g., in memory 420) the sensed signal as a baseline. More specifically, at least a pair of sensing electrodes (e.g., 326, 327) can be used to perform step 202. In accordance with certain embodiments, both electrodes of a pair of sensing electrodes (e.g., 326, 327) are located on the catheter (e.g., 302). In other embodiments, one sensing electrode of a pair of sensing electrodes is located on the catheter (e.g., 302), while another sensing electrode of the pair is located in the distal end of an introducer sheath (that is used to insert the catheter into the biological lumen), or on the distal end of a guide wire (that is used to guide the catheter into the biological lumen), or alternatively is an external skin electrode located on the skin of a patient. In accordance with certain embodiments, this baseline neural activity can be used (e.g., by the controller 422) to diagnose an autonomic disease state, such as, but not limited to, chronic hypertension. Step 204 involves setting (e.g., by the controller 422) a stimulation level to a low (e.g., minimum) setting, and step 206 involves emitting stimulation (e.g., by the signal generator or stimulator 402) at the low setting via one or more stimulation electrodes (e.g., 324, 325) of the catheter (e.g., 302). Step 208 involves sensing (e.g., by the sensing circuit 404) neural activity, using sensing electrodes (e.g., 326, 327), at least one of which is on a catheter (e.g., 302), within a window (temporal window) following the stimulation emitted at step 206. At step 210 the neural activity sensed (e.g., by the sensing circuit 404) at step 208 is compared (e.g., by the controller 422) to the baseline neural activity sensed at step 202 to determine whether stimulation emitted at the set level caused an evoked neural response. At step 212, based on a result of the comparison performed at step 210, there is a determination (e.g., by the controller 422) of whether an evoked neural response occurred, because an evoked neural response is distinguishable from spontaneous neural activity. For an example, if there is an increase in the neural activity (compared to spontaneous neural activity) by at least a specified threshold, then it can be concluded (e.g., by the controller 422) that an evoked neural response occurred. If the answer to the determination at step 212 is No, then the stimulation level setting is increased (e.g., by the controller 422) at step 214, and then flow returns to step 206 and stimulation is emitted at the increased level at step 206. Steps 208, 210, and 212 are then repeated (as may step 214) until the answer to the determination at step 212 is Yes, at which point flow goes to step 216. At step 216 the minimum stimulation level needed to evoke a neural response is saved (e.g., by the controller 422) in memory (e.g., 420 in FIG. 4), and at step 218 the evoked neural response signal (and more specifically, data indicative thereof) is preferably also stored in memory (e.g., 420 in FIG. 4).

At step 220 the stimulation level setting is increased (e.g., by the controller 422) and the stimulation energy is emitted at the increased stimulation level, and at step 222 the evoked neural response signal to the stimulation at the increased level is stored in memory. It is noted that whenever it is stated that a signal is stored, it can be an analog version of the signal, but more likely is digital samples of the signal, or more generally data indicative of the signal, such as data specifying the maximum amplitude of the signal, an area under the curve of the signal, and/or data indicative of the morphology of the signal, but not limited thereto.

At step 224 the stimulation level setting is again increased (e.g., by the controller 422) and the stimulation energy (e.g., generator by the signal generator 406) is emitted at the increased stimulation level, and at step 226 the evoked neural response signal that is responsive to the stimulation at the increased level is stored (e.g., in the memory 420). At step 228 the evoked neural activity sensed at the two most recently tested stimulation levels are compared to one another (e.g., by the controller 422) to determine whether the evoked neural response has saturated, i.e., reached its maximum. At step 230 based on a result of the comparison performed at step 228 there is a determination (e.g., by the controller 422) of whether saturation occurred. If the answer to the determination is No, then the stimulation level setting is increased (e.g., by the controller 422) at step 224, and then steps 226, 228, and 230 are repeated until the answer to the determination (e.g., by the controller 422) at step 230 is Yes, at which point flow goes to step 232. At step 232 the stimulation level at which saturation of the evoked neural response occurred is stored (e.g., in the memory 420). The information stored (e.g., by the controller 422) at step 232 and/or at step 216 (e.g., in the memory 422) are further examples of characteristics of sensed neural activity that can be determined at step 106 in FIG. 1, and used (e.g., by the controller 422) to select denervation parameters at step 108 in FIG. 1.

In certain embodiments, the greater the minimal amount of stimulation energy that is needed to evoke a neural response, the greater the amount of ablation energy that is needed to denervate the nerves of interest. For example, where RF energy is used to perform nerve ablation, the RF energy level can be set to some multiple (e.g., 3x, or 5x, but not limited thereto) of the minimal amount of stimulation energy that is needed to evoke a neural response. For another example, where ultrasound is used to perform nerve ablation, the greater the minimal amount of stimulation energy that is needed to evoke a neural response, the greater the magnitude of ultrasound energy that is used to perform nerve ablation, wherein the magnitude can be in terms of amplitude and/or temporal duration, but is not limited thereto. This is in part because the greater the minimal amount of stimulation energy that is needed to evoke a neural response, likely the greater the distance of the nerves from the sensing and treatment location.

In accordance with certain embodiments, the emission of RF energy from electrodes (e.g., 324, 325) of a catheter (e.g., 302) are focusable, i.e., the electrodes are configured as a focused array that can be used to focus RF energy at a specific distance or depth relative to the electrodes. Accordingly, where the nerves that are to be ablated are relatively shallow (i.e., relatively close a wall of the biological lumen within which a catheter is inserted), the RF energy can be focused to a relatively shallow depth. Conversely, where the nerves that are to be ablated are relatively deep (i.e., relatively far from a wall of the biological lumen), the RF energy can be focused to a greater depth. Indicators of the relative depth of the nerves are examples of characteristics of neural activity that can be determined (e.g., by the controller 422) at step 106 in FIG. 1, and the depth at which RF energy is focused is an example of a denervation parameter that can be selected (e.g., by the controller 422) at step 108 in FIG. 1, and used at step 110 in FIG. 1, in accordance with certain embodiments of the present technology.

As will be described in additional detail below, where an ultrasound transducer is used to emit ultrasound energy to perform denervation, the ultrasound transducer (e.g., 311) can be located on a catheter (e.g., 302) within a balloon (e.g., 313) which is at least partially filled with a cooling fluid (e.g., 613) that is circulated through the balloon. Such cooling fluid (e.g., 613) an be used to cool and protect a wall of a biological lumen within which the balloon (e.g., 313) and ultrasound transducer (e.g., 311) are located. In general, the greater the flow rate of the cooling fluid through the balloon, the greater the depth of the protection provided by the cooling fluid. Similarly, if the temperature of the cooling fluid is controllable, e.g., using a cooling mechanism, such as a cooling coil, then the lower the temperature of the cooling fluid the greater the depth of protection provided by the cooling fluid. Accordingly, where the nerves that are to be ablated are relatively shallow (i.e., relatively close a wall of the biological lumen within which the balloon and ultrasound transducer are located), a relatively slow cooling fluid flow rate may be selected (e.g., by the controller 422), and/or the temperature of the cooling fluid need not be very cold (i.e., need not be very low). Conversely, where the nerves that are to be ablated are relatively deep (i.e., relatively far from a wall of the biological lumen), a relatively fast cooling fluid flow rate may be selected (e.g., by the controller 422), and/or the temperature of the cooling fluid should be made cooler (i.e., lower). Indicators of the relative depth of the nerves are examples of characteristics of neural activity that can be determined (e.g., by the controller 422) at step 106 in FIG. 1, and the flow rate of a cooling fluid and/or a controlled temperature thereof are examples of denervation parameters that can be selected (e.g., by the controller 422) at step 108 in FIG. 1, and used at step 110 in FIG. 1, in accordance with certain embodiments of the present technology.

As noted above, in certain embodiments, baseline spontaneous (aka native) neural activity can be used (e.g., by the controller 422) to diagnose an autonomic disease state, such as, but not limited to, chronic hypertension. Alternatively, or additionally, an evoked neural response to electrical stimulation can be used (e.g., by the controller 422) to diagnose an autonomic disease state, such as, but not limited to, chronic hypertension. An evoked neural response can alternatively be responsive to (i.e., evoked in response to) some other types of stimulation besides electrical stimulation, such as responsive to one or more physical maneuvers, and/or responsive to administration of a drug bolus, but not limited thereto. More generally, neural activity and changes thereto can be observed (e.g., by the controller 422) to quantify health of nerves, and because unhealthy nerves should have recognizable neural conduction patterns, sensed neural activity can be used (e.g., by the controller 422) to diagnose a root cause of a disease state, in accordance with certain embodiments of the present technology.

Embodiments of the present technology can be implemented using various different catheter implementations, and various implementations of an electronic control unit (ECU), and thus, are not limited to use with any specific catheter, ECU, and/or system of which a catheter and/or ECU is a part. Nevertheless, for completeness, an example catheter 302, ECU 402 and system 400 that can be used to implement embodiments of the present technology are described below. More specifically, FIGS. 3A through 7B are used to describe an example catheter 302, ECU 402 and system 400 that can be used to implement embodiments of the present technology that were described above. Such a system 400 can also be referred to as an apparatus or device herein.

### EXAMPLE CATHETER

FIG. 3A shows a catheter 302 with its selectively deployable electrodes 324 and 326 in their non-deployed positions. The catheter 302 includes a catheter handle 312 and a catheter shaft 322. In addition to including the selectively deployable electrodes 324 and 326, the catheter shaft 322 is also shown as including a non-deployable electrode 325 that is proximal to the selectively deployable electrode 324, and a non-deployable electrode 327 that is distal the selectively deployable electrode 326. The selectively deployable electrode 324 can also be referred to as the proximal selectively deployable electrode 324, or more succinctly as the proximal electrode 324, or even more succinctly as the electrode 324. The selectively deployable electrode 326 can also be referred to as the distal selectively deployable electrode 326, or more succinctly as the distal electrode 326, or even more succinctly as the electrode 326. The catheter shaft 322 can also be referred to more succinctly herein as the shaft 322. The catheter 302 can be a specific implementation of the catheter 302 shown in and discussed above with reference to FIG. 3.

The catheter shaft 322 is also shown as including a balloon 313 positioned longitudinally between the electrodes 324 and 326, wherein the balloon 313 is selectively inflatable and deflatable. The balloon 313 can also be referred as a selectively inflatable balloon 313, a selectively deployable balloon 313, or more succinctly as a balloon 313. When the balloon 313 is deflated, it can also be referred to as being non-inflated or in its non-deployed position. When the balloon 313 is inflated, it can also be referred to as being in its deployed position. As will be described in additional detail below, the balloon 313 can be selectively inflated by injecting a fluid into the balloon 313, and the balloon 313 can be selectively deflated by removing the fluid from the balloon 313. The balloon 313 can be made of an electrically insulating material such as polyamide, polyethylene terephthalate, or thermoplastic elastomer. In specific embodiments the balloon 313 is made from nylon, a polyimide film, a thermoplastic elastomer (such as those marked under the trademark PEBAX™), a medical-grade thermoplastic polyurethane elastomers (such as those marketed under the trademark PELLETHANE™), pellethane, isothane, or other suitable polymers or any combination thereof, but is not limited thereto.

The catheter handle 312, which can also be referred to more succinctly as the handle 312, includes actuators 314, 316, and 318, which can be used to selectively deploy the electrodes 324, 326, as well as to adjust a longitudinal distance between the electrodes 324, 326, as will be described in additional detail below. The actuators 314, 316, and 318 are respectively slidable within slots 315, 317, and 319 in the handle 312, and thus, the actuators 314, 316, and 318 can also be referred to as sliders. The catheter handle 312 is also shown as including a fluidic inlet port 334a and a fluidic outlet port 334b.

A fluid (e.g., expelled from a pressure syringe) can enter a fluid lumen (in the catheter shaft 322), via the fluidic inlet port 334a of the catheter 302, and then enter and at least partially fill the balloon 313. Fluid can be drawn from the balloon 313 (e.g., using a vacuum syringe) through another fluid lumen (in the catheter shaft 322) and out the fluidic outlet port 334b of the catheter 302. In this manner, the fluid can be used to selectively inflate and selectively deflate the balloon 313. In certain embodiments, fluid can be simultaneously injected into and removed from the balloon 313 to thereby circulate the fluid through the balloon 313.

The catheter 302 can also be referred to as an intraluminal microneurography probe 302, or more succinctly, as a probe 302. A cable 304, which extends from a proximal portion of the handle 312, provides for electrical connections between the catheter 302 (and more specifically, the electrodes thereof) and an electrical control unit (ECU) (e.g., 402), an example of which is described below with reference to FIG. 4.

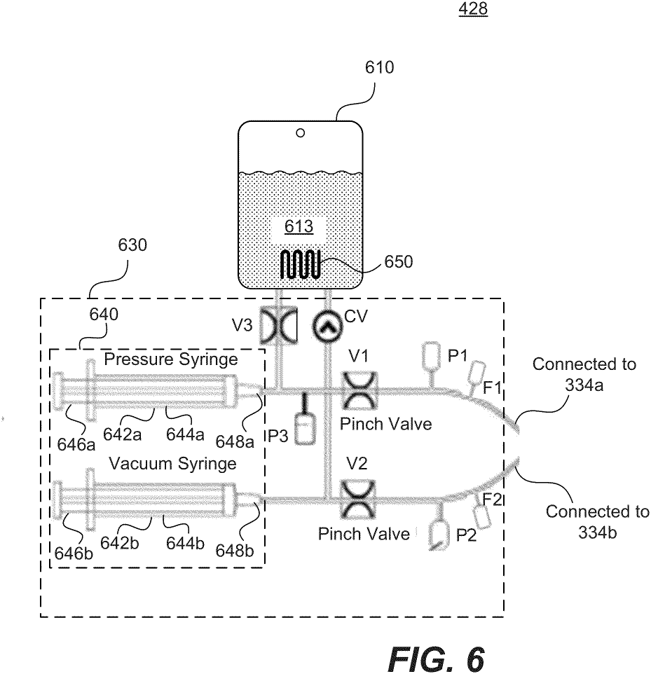

Still referring to FIG. 3A, a transducer 311 is shown as being within a balloon 313. The transducer 311 is an example of an ablation element that is included on the shaft 322 and configured to ablate nerve tissue using ultrasound energy. In other embodiments, the transducer and balloon may be replaced by a helical structure carrying a plurality of electrodes configured to deliver RF and/or pulsed electric field RF energy. In other embodiments, the transducer and balloon may be replaced by a microwave transmitting element within an expandable centering element. In other embodiments, the transducer may be replaced by a cryotherapeutic applicator. In other embodiments, the transducer and balloon may be replaced by an infusion needle configured to deliver an ablative chemical to the renal nerves.

Where a transducer 311 is within the balloon 313, the fluid that is circulated through the balloon 313 can be referred to as a cooling fluid that is used to cool the transducer 311 and/or to cool a portion of a biological lumen that the balloon 313 is within, and/or to cool the biological tissue surrounding the lumen. It is also possible that the catheter 302 is devoid of the transducer 311 or other ablative means, and that a separate catheter that includes a transducer or other ablative means is used to deliver ablation energy (e.g., at step 602 and 604 in FIG. 6). Where the catheter 302 is devoid of the transducer 311 or other ablative means, one or more electrodes of the catheter 302 can be used for sensing native neural activity. One or more electrodes of the catheter 302 can be used for delivering stimulation energy and one or more further electrodes of the catheter 302 can be used for sensing an evoked neural response to the stimulation energy.

When the catheter 302 is inserted into a biological lumen, such as an artery, vein or other vasculature, it is the distal portion of the catheter 302 (and more specifically the shaft 322) that is inserted into the biological lumen, and the proximal end of the catheter 302 (and more specifically the handle 312) that is used to maneuver the catheter 302. In the embodiment shown in FIGS. 3A and 3B, the electrode 326 can also be referred to as a distal selectively deployable electrode 326 as noted above, since it located closer to the distal end of the catheter 302 than to the proximal end of the catheter 302; and the electrode 324 can also be referred to as a proximal selectively deployable electrode 324 as noted above, since it is located closer to the proximal end of the catheter 302 than to the distal end of the catheter 302. For similar reasons, the electrode 325 can be referred to as the proximal non-deployable electrode 325, and the electrode 327 can be referred to as the distal non-deployable electrode 327.

FIG. 3B shows the catheter 302 with the electrodes 324 and 326 in their deployed (aka expanded) positions. In certain embodiments, the proximal selectively deployable electrode 324 is configured to be deployed (aka expanded) in response to the actuator 314 being slid in the proximal direction indicated by the arrow 344 in FIG. 3B. In such an embodiment, the proximal electrode 324 can be returned to its non-deployed (aka non-expanded or retracted) position in response to the actuator 314 being slid in the distal direction opposite the arrow 344 in FIG. 3B. More generally, the actuator 314 is used to selectively expand and retract the electrode 324.

In accordance with certain embodiments, the longitudinal distance between the distal electrode 326 and the proximal electrode 324 can be reduced by sliding the actuator 318 in the proximal direction indicated by the arrow 348 in FIG. 3B. Thereafter, the longitudinal distance between the distal electrode 326 and the proximal electrode 324 can be increased, if desired, by sliding the actuator 318 in the distal direction opposite the arrow 348 in FIG. 3B. More generally, the actuator 318 is used to adjust the longitudinal distance between the electrodes 324 and 326. The longitudinal distance between the proximal and distal electrodes 324, 326 can be any distance between the maximum and minimum longitudinal distance as controlled by a user using the actuator 318. In accordance with certain embodiments, the electrode 324 is configured to be deployed in response to the actuator 314 being slid in the proximal direction indicated by the arrow 344 in FIG. 3B. In accordance with certain embodiments, the distal electrode 326 is configured to be deployed in response to the actuator 316 being slid in the proximal direction indicated by the arrow 346 in FIG. 3B. In such an embodiment, the distal electrode 326 can be returned to its non-deployed position in response to the actuator 316 being slid in the distal direction opposite the arrow 346 in FIG. 3B. More generally, the actuator 316 is used to selectively expand and retract the electrode 326. Other variations are also possible and within the scope of the embodiment described herein.

Each of the selectively deployable electrodes 324, 326 can be made, for example, of a unitary nitinol tube that is laser cut to include apertures or openings having a predetermine pattern. In FIGS. 3A and 3B, each of the electrodes 324, 326 has laser cut spiral apertures that extend between proximal and distal portions of each of the electrodes 324, 326. The spiral apertures in each of the electrodes 324, 326 enable each of the electrodes to be selectively transitioned between their non-deployed and deployed positions. The apertures that are cut into the electrodes 324, 326 can have other shapes besides being spiral, so long as the apertures enable the electrodes to be transitioned between non-deployed and deployed positions. The selectively deployable electrodes 324, 326 can alternatively be mesh electrodes or spiral electrodes made of one or more electrically conductive wires, optionally with portions thereof being insulated. Other variations are also possible and within the scope of the embodiments described herein.

The catheter 302 can be configured to be introduced into a biological lumen, such as an artery, in a location near a body organ, such as a kidney. The catheter 302 can be introduced via an introducer sheath that is advanced to the intended catheter location in the biological lumen, and then withdrawn sufficiently to expose the shaft 322 to the biological lumen (e.g., renal artery). Once the shaft 322 is within the biological lumen, one of the electrodes 324, 326 can be deployed (aka expanded) using one of the actuators 314, 316 such that it is in contact with a portion of a circumferential interior wall of the biological lumen. The longitudinal distance between the electrodes 324 and 326 can then be adjusted, if desired, using the actuator 318. The other one of the electrodes 324, 326 can then be deployed (aka expanded) such that it is in contact with another portion of the circumferential interior wall of the biological lumen.

For example, where the catheter 302 is inserted into a renal artery close to a kidney, the electrodes can be positioned near a nerve bundle that connects the kidney to the central nervous system, as the nerve bundle tends to approximately follow the artery leading to most body organs. The nerve bundle tends to follow the artery more closely at the end of the artery closer to the kidney, while spreading somewhat as the artery expands away from the kidney. As a result, it is desired in some examples that the catheter shaft 322 is small enough to introduce relatively near the kidney or other organ, as nerve proximity to the artery is likely to be higher nearer the organ.

Once the catheter 302 is in place, a practitioner can use instrumentation (e.g., the ECU 402) coupled to the electrodes to stimulate one or more nerves, and monitor for evoked nerve response signals used to characterize the nervous system response to certain stimulus. The transducer 311 and/or other ablative means are configured to ablate nerve tissue, such as by using ultrasound, RF, pulsed electric field RF, microwave, cryotherapy, or other energy or chemical means. Additionally, the catheter 302 can actively stimulate one or more nerves and sense resulting neural signals in between applications of ablation energy via the transducer 311, enabling more accurate control of the degree and effects of nerve ablation. In other examples, a catheter 302 lacking a transducer or other ablative means can be removed via a sheath, and an ablation probe (aka catheter) inserted, with the ablation probe removed and the catheter 302 reinserted to verify and characterize the effects of the ablation probe.

Any one or more electrodes of the catheter 302 can be selectively used to deliver stimulation energy to nerves surrounding a biological lumen. Similarly, any one or more electrodes of the catheter can be selectively used to sense neural activity of nerves surrounding a biological lumen, which can be spontaneous neural activity, or evoked neural activity.

For much of the below discussion, it is assumed that the transducer 311 is an ultrasound transducer that can be activated to deliver unfocused ultrasound energy radially outwardly so as to suitably heat, and thus treat, tissue within the target anatomical region. The transducer 311 can be activated at a frequency, duration, and energy level suitable for treating the targeted tissue. In one nonlimiting example, the unfocused ultrasound energy generated by the transducer 311 may target select nerve tissue of the subject, and may heat such tissue in such a manner as to neuromodulate (e.g., fully or partially ablate, necrose, or stimulate) the nerve tissue.

In accordance with certain embodiments, the transducer 311 includes a piezoelectric transducer body that comprises a hollow tube of piezoelectric material having an inner surface and an outer surface, with an inner electrode disposed on the inner surface of the hollow tube of piezoelectric material, and an outer electrode disposed on the outer surface of the hollow tube of piezoelectric material. In such embodiments, the hollow tube of piezoelectric material is an example of the piezoelectric transducer body. The hollow tube of piezoelectric material, or more generally the piezoelectric transducer body, can be cylindrically shaped and have a circular radial cross-section. However, in alternative embodiments the hollow tube of piezoelectric material can have other shapes besides being cylindrical with a circular radial cross-section. Other cross-sectional shapes for the hollow tube of piezoelectric material, and more generally the piezoelectric transducer body, include, but are not limited to, an oval or elliptical cross-section, a square or rectangular cross-section, pentagonal cross-section, a hexagonal cross-section, a heptagonal cross-section, an octagonal cross-section, and/or the like. The hollow tube of piezoelectric material, and more generally the piezoelectric transducer body, can be made from various different types of piezoelectric material, such as, but not limited to, lead zirconate titanate (PZT), polyvinylidene fluoride (PVDF), or other presently available or future developed piezoelectric ceramic materials. In other embodiments, the transducer 311 can be made of other materials and/or can have other shapes.

In certain embodiments, the transducer 311 is an ultrasound transducer configured to deliver acoustic energy in the frequency range of 8.5 to 9.5 MHz. In certain embodiments, the transducer is configured to deliver acoustic energy in the frequency range of 8.7-9.3 MHz or 8.695-9.304 MHz. Transducers delivering acoustic energy in the frequency range of 8.7-9.3 MHz have been shown to produce ablation up to mean depths of 6 mm. The piezoelectric transducer body is configured to generate ultrasound waves in response to a voltage being applied between the inner and outer electrodes. One or both of the inner and outer electrodes can be covered by an electrical insulator to inhibit (and preferably prevent) a short circuit from occurring between the inner and outer electrodes when the ultrasound transducer is placed within an electrically conductive fluid and a voltage is applied between the inner and outer electrodes. Such an electrical insulator can be parylene, and more specifically, a parylene conformal coating, but is not limited thereto. An excitation source (e.g., 426 in FIG. 4) may be electrically coupled to inner and outer electrodes of the transducer 311, and may actuate the transducer 311 by applying a voltage between the inner and outer electrodes (or any other pair of electrodes), so as to cause the piezoelectric material of the piezoelectric transducer body to generate an unfocused ultrasound wave that radiates radially outwardly.

### EXAMPLE ELECTRICAL CONTROL UNIT (ECU)

FIG. 4 is a high level block diagram of an electrical control unit (ECU) 402 that is configured to be in electrical communication with a catheter, such as the catheter 302 described above. The ECU 402, and the catheter (e.g., 302) to which the ECU 402 is electrically coupled via a cable (e.g., 304), can be referred to more generally as a system 400. The ECU 402 can process a received signal to produce an output signal, and present information including information about the output signal, the received signal, or processing information. Such a system 400 can be used, for example, in diagnostic procedures for assessing the status of a patient’s nervous activity proximate a biological lumen, such as a vein or an artery, e.g., a renal artery, or another type of blood vessel. Such a system 400 can be additionally, or alternatively, be used to select preferred denervation parameters for use in a denervation procedure. A same catheter (e.g., 302) that is used to assess the status of a patient’s nervous activity proximate and/or select preferred denervation parameters can also be used to perform a denervation procedure. Alternatively, it is possible that the catheter that is used to perform a denervation procedure differs from the catheter (e.g., 302) that is used to assess that status of a patient’s nervous activity proximate a biological lumen, in which case different catheters can be swapped in and out of a biological lumen during a procedure.

Still referring to FIG. 4, the ECU 402 includes a stimulator 406 electrically coupled to a selected pair of electrodes (e.g., 324 and 325) of the catheter 302. The stimulator 406 which is part of a STIM circuit or subsystem 404, can selectively emit electrical signals (including stimulation pulses) having a specific voltage, amperage, duration, duty cycle and/or frequency of application that will cause nerve cell activation. For an example, the electrode 327 can be connected as the stimulation anode and the electrode 326 can be connected as the stimulation cathode, or vice versa. For another example, the electrode 324 can be connected as the stimulation anode and the electrode 325 can be connected as the stimulation cathode, or vice versa. Switches, which are not specifically shown, can be used to selectively control how various electrodes (e.g., 324, 325, 326 and 326) are coupled to various nodes of the ECU 402, such as to the input terminals of the amplifier 412, or to the output terminals of the stimulator 406. In this manner, the switches can be used to control which electrodes are configured as stimulation electrodes and which electrodes are configured as sensing electrodes. The stimulator 406 can also be referred to herein as a signal generator 406.

Upon receiving the stimulation signal produced by the stimulator 406, the electrodes of the catheter 302 that are connected as stimulation electrodes (e.g., 324 and 325) can apply electrical energy to a patient’s nerves through the biological lumen wall based on the received signal. Such stimulus can have any of a variety of known waveforms, such as a sinusoid, a square wave form or a triangular wave form, but is not limited thereto. In various examples, the stimulation can be applied for durations between approximately 0.05 milliseconds (msec) and approximately 8 msec.

The stimulation of nerves can be performed to evoke an elicited potential, which can cause such a potential to propagate in every direction along the nerve fibers. More generally, the STIM subsystem 405 can be used to deliver electrical stimulation via a selected pair of electrodes in order to evoke a neural response, and the SENS subsystem 404 can be used to sense the evoked neural response.

In some embodiments, the ECU 402 can digitally sample the signal sensed using a pair of electrodes (e.g., 326, 327) to receive the electrical signal from the catheter (e.g., 302). In alternate embodiments, the signal can be recorded as an analog signal. When receiving an electrical signal from the electrodes (e.g., 326, 327) on the catheter (e.g., 302), the ECU 402 can perform filtering and/or other processing steps on the signal. Generally, such steps can be performed to discriminate the signal sensed by the catheter (e.g., 302) from any background noise within the patient’s vasculature such that the resulting output is predominantly the signal from nerve cell activation. In some instances, the ECU 402 can modulate the electrical impedance of the signal receiving portion in order to accommodate the electrical properties and spatial separation of the electrodes (e.g., 326, 327) mounted on the catheter (e.g., 302) in a manner to achieve the highest fidelity, selectively and resolution for the signal received. For example, electrode size, separation, and conductivity properties can impact the field strength at the electrode/tissue interface.

Additionally or alternatively, the ECU 402 can comprise a headstage and/or an amplifier to perform any of offsetting, filtering, and/or amplifying the signal received from the catheter. In some examples, a headstage applies a DC offset to the signal and performs a filtering step. In some such systems, the filtering can comprise applying notch and/or band-pass filters to suppress particular undesired signals having a particular frequency content or to let pass desired signals having a particular frequency content. An amplifier can be used to amplify the entire signal uniformly or can be used to amplify certain portions of the signal more than others. For example, in some configurations, the amplifier can be configured to provide an adjustable capacitance of the recording electrode, changing the frequency dependence of signal pick-up and amplification. In some embodiments, properties of the amplifier, such as capacitance, can be adjusted to change amplification properties, such as the resonant frequency, of the amplifier.

In the illustrated embodiment of FIG. 4, the ECU 402 includes an amplifier 412 including a non-inverting (+) input terminal, an inverting (-) input terminal, a power supply input terminal, and a ground or reference terminal. As can be appreciated from FIG. 4, the non-inverting (+) input terminal can be coupled to the electrode 326, the inverting (-) input terminal can be electrically coupled to the electrode 327, the power supply input terminal is electrically coupled to a voltage source (e.g., a reference voltage generator), and the ground or reference terminal is electrically coupled to a ground reference electrode, which can be located on the catheter 302, can be located on a distal end of an introducer sheath, or can be located on the skin of the patient, but is not limited thereto.

In some embodiments, the ECU 402 can include a switching network configured to interchange which of electrodes of a catheter (e.g., 302) are coupled to which portions of the ECU. In some such embodiments, a user can manually switch which inputs receive connections to which electrodes of the catheter 302. Such configurability allows for a system operator to adjust the direction of propagation of the elicited potential as desired. For example, the switching network, or more generally switches, can be used to connect the electrodes 324 and 325 to the stimulator 406 during a period of time during which stimulation pulses are to be emitted by the catheter 302, and the switches can be used to connect the electrodes 326 and 326 to the amplifier 412 to sense the elicited response to the stimulation pulses. Additionally, or alternatively, a controller (e.g., 422) can autonomously control such a switching network.

The amplifier 412 can include any appropriate amplifier for amplifying desired signals or attenuating undesired signals. In some examples, the amplifier has a high common-mode rejection ratio (CMRR) for eliminating or substantially attenuating undesired signals present in each at each of the sensing electrodes (e.g., 326 and 327). In some embodiments, the amplifier 412 can be adjusted, for example, via an adjustable capacitance or via other attributes of the amplifier 412.

In the example system 400 of FIG. 4, the ECU 402 further includes a filter 414 for enhancing the desired signal in the signal received via a pair of the electrodes. The filter 414 can include a band-pass filter, a notch filter, or any other appropriate filter to isolate desired signals from noise artifacts within the received signals. In some embodiments, various properties of the filter 414 can be adjusted to manipulate its filtering characteristics. For example, the filter may include an adjustable capacitance or other parameter to adjust its frequency response.

At least one of amplification and filtering of a sensed signal (e.g., received at the electrodes 326 and 327) can allow for extraction of the desired signal at or by the extraction module 416. In some embodiments, extraction performed by the extraction module 416 comprises at least one additional processing step to isolate desired signals from the signal sensed using electrodes such as preparing the signal for output at 418. In some embodiments, the functionalities of any combination of amplifier 412, filter 414, and extraction module 416 may be combined into a single entity. For instance, the amplifier 412 may act to filter undesired frequency content from the signal without requiring additional filtering at a separate filter.

In some embodiments, the ECU 402 can record emitted stimuli and/or received signals. Such data can be subsequently stored in permanent or temporary memory 420. The ECU 402 can comprise such memory 420 or can otherwise be in communication with external memory (not shown). Thus, the ECU 402 can be configured to emit stimulus pulses to electrodes of the catheter, record such pulses in a memory, receive signals from the catheter, and also record such received signal data. The memory 420 in or associated with the ECU 402 can be internal or external to any part of the ECU 402 or the ECU 402 itself.

The ECU 402 or separate external processor can further perform calculations on the stored data to determine characteristics of signals either emitted or received via the catheter. For example, in various embodiments, the ECU 402 can determine any of the amplitude, duration, or timing of occurrence of the received or emitted signals. The ECU 402 can further determine the relationship between the received signal and the emitted stimulus signal, such as a temporal relationship therebetween. In some embodiments, the ECU 402 performs signal averaging on the signal data received from the catheter. Such averaging can act to reduce random temporal noise in the data while strengthening the data corresponding to any elicited potentials received by the catheter.

Averaging as such can result in a signal in which temporally random noise is generally averaged out and the signal present in each recorded data set, such as elicited potentials, will remain high. In some embodiments, each iteration of the process can include a synchronization step so that each acquired data set can be temporally registered to facilitate averaging the data. That is, events that occur consistently at the same time during each iteration may be detected, while temporally random artifacts (e.g., noise) can be reduced. In general, the signal to noise ratio (SNR) resulting in such averaging will improve by the square root of the number of samples averaged in order to create the averaged data set.

The ECU 402 can further present information regarding any or all of the applied stimulus, the signal, and the results of any calculations to a user of the system, e.g., via output 418. For example, the ECU 402 can generate a graphical display providing one or more graphs of signal strength vs. time representing the stimulus and/or the received signal.

In some embodiments, the ECU 402 can include a controller 422 in communication with one or both of stimulator 406 and SENS subsystem 404. The controller 422 can be configured to cause stimulator 406 to apply a stimulation signal to a catheter, e.g., the catheter 302. Additionally or alternatively, the controller 422 can be configured to analyze signals received and/or output by the SENS subsystem 404. In some embodiments, the controller 422 can act to control the timing of applying the stimulation signal from stimulator 406 and the timing of receiving signals by the SENS subsystem 404. The controller 422 can be implemented, e.g., using one or more processors, field programmable gate arrays (FPGAs), state machines, and/or application specific integrated circuits (ASICs), but is not limited thereto.

Example electrical control units have been described. In various embodiments, the ECU 402 can emit stimulus pulses to the catheter 302, receive signals from the catheter 302, perform calculations on the emitted and/or received signals, and present the signals and/or results of such calculations to a user. In some embodiments, the ECU 402 can comprise separate modules for emitting, receiving, calculating, and providing results of calculations. Additionally or alternatively, the functionality of controller 422 can be integrated into the ECU 402 as shown, or can be separate from and in communication with the ECU.

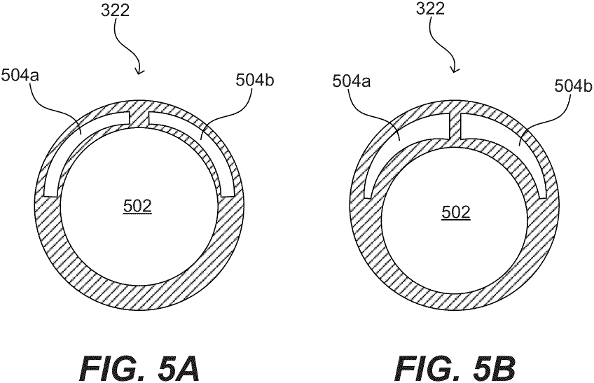

The controller 422 can also control a fluid supply subsystem 428, which can include a cartridge and a reservoir, which are described below with reference to FIG. 6, but can include alternative types of fluid pumps, and/or the like. The fluid supply subsystem 428 is fluidically coupled to one or more fluid lumens (e.g., 504a, 504b, in FIGS. 5A, 5B) within the catheter shaft 322 which in turn are fluidically coupled to the balloon 313. The fluid supply subsystem 428 can be configured to circulate a cooling liquid through the catheter 302 to the transducer 311 in the balloon 313.

In some embodiments, the ECU 402 can include an excitation source 426 in FIG. 4 may be electrically coupled to inner and outer electrodes of the transducer 311, and may actuate the transducer 311 by applying a voltage between the inner and outer electrodes (or any other pair of electrodes), so as to cause the piezoelectric material of the piezoelectric transducer body to generate an unfocused ultrasound wave that radiates radially outwardly.

In an embodiment, the tissue treatment system 400 comprises a signal generator, such as a stimulator 406 and/or an ultrasound excitation source 426. The system 400 also comprises a sensing circuit 404 coupleable to at least one electrode 326, 327 of a catheter 302 that is insertable into a biological lumen. The sensing circuit 404 is configured to sense neural activity of nerves within tissue surrounding the biological lumen using the at least one electrode 326, 327 of the catheter 302 while the catheter 302 is inserted into the biological lumen. The system 400 further comprises a controller 422 communicatively coupled to the signal generator 406, 426 and the sensing circuit 404. The controller 406 comprises one or more processors and configured to determine one or more characteristics of the sensed neural activity of the nerves within the tissue surrounding the biological lumen. The one or more characteristics are indicative of one or more of a size, type, function or health of the nerves and/or indicative of proximity of the nerves relative to the at least one electrode 326, 327 of the catheter 302. The one or more processors of the controller 422 are also configured to select one or more denervation parameters based on the one or more characteristics of the sensed neural activity and control the signal generator 406, 426 to generate, using the selected one or more denervation parameters, signals for performing a denervation procedure intended to denervate at least some of the nerves for which the neural activity was sensed.

In an embodiment, the system 400 also comprises memory 420 configured to store instructions executable by the one or more processors of the controller 422 to perform the above-mentioned determination, selection and control operations.

In an embodiment, the system 400 comprises the catheter 302. In this embodiment, the sensing circuit 404 is configured to sense the neural activity of the nerves using the at least one electrode 326, 327, and the signal generator 406, 426 is configured to generate the signals for performing the denervation procedure using at least one other electrode 324, 325 of the catheter 302, or an ultrasound transducer 311 of the catheter 302 configured to emit ultrasound energy.

In an embodiment, the sensing circuit 404 is configured to sense evoked neural activity of the nerves within the tissue surrounding the biological lumen using the at least one electrode 326, 327 of the catheter 302. In this embodiment, the evoked neural activity is responsive to electrical stimulation delivered using the at least one other electrode 324, 325 of the catheter 302.

In an embodiment, the system 400 comprises a first catheter 302 comprising the at least one electrode 326, 327, and a second catheter 302 comprising at least one electrode 324, 325 and/or an ultrasound transducer 311 configured to emit ultrasound energy. In this embodiment, the sensing circuit 404 is configured to sense the neural activity of the nerves using the at least one electrode 326, 327 of the first catheter 302. The signal generator 406, 426 is, in this embodiment, configured to generate the signals for performing the denervation procedure using the at least one electrode 324, 325 of the second catheter 302, or the ultrasound transducer 311 of the second catheter 302.

In an embodiment, the one or more denervation parameters selected by the controller 422 comprise one or more of amplitude, power, duration, frequency, and duty cycle of the ultrasound energy of the ultrasound transducer 311.

In an embodiment, the ultrasound transducer 311 is located within a balloon 313 that is at least partially filled with a cooling fluid that is circulated through the balloon 313 in order to cool at least a portion of the tissue surrounding the biological lumen proximate the balloon 313 The one or more denervation parameters selected by the controller 422 also comprise at least one of a flow rate or a temperature associated with the cooling fluid.

In an embodiment, the denervation parameters selected by the controller 422 comprise one or more of amplitude, power, duration, frequency, and duty cycle of radio frequency (RF) energy emitted by the at least one other electrode 324, 325 of the catheter 302 or the at least one electrode 324, 325 of the second catheter 302.

In an embodiment, the controller 422 is configured to determine at least one of the one or more characteristics of the sensed neural activity by determining a minimal amount of stimulation energy needed to evoke a neural response by the nerves within tissue surrounding the biological lumen. The controller 422 is also configured to select the one or more denervation parameters based on the one or more characteristics of the sensed neural activity by selecting at least one of the one or more denervation parameters based on the minimal amount of stimulation energy needed to evoke the neural response by the nerves within tissue surrounding the biological lumen.

In an embodiment, the controller 422 is configured to determine at least one of the one or more characteristics of the sensed neural activity by determining an amount of stimulation energy needed to cause saturation of an evoked neural response of the nerves within tissue surrounding the biological lumen. The controller 422 is also configured to select the one or more denervation parameters based on the one or more characteristics of the sensed neural activity by selecting at least one of the one or more denervation parameters based on the amount of stimulation energy needed to cause saturation of the evoked neural response of the nerves within tissue surrounding the biological lumen.

In an embodiment, the controller 422 is configured to select at least one of the one or more denervation parameters using one or more tables accessed by the controller 422 from a memory 420.

In an embodiment, the controller 422 is configured to select at least one of the one or more denervation parameters using a machine learning model implemented by at least one of the one or more processors of the controller 422.

In an embodiment, the sensing circuit 404 is configured to sense the neural activity of the nerves within the tissue surrounding the biological lumen by sensing a signal indicative of the neural activity, the signal indicative of the neural activity including multiple peaks temporally spaced apart from one another. The controller 422 is configured to determine at least one of the one or more characteristics of the sensed neural activity based on at least one of amplitudes of the multiple peaks or based on temporal spacings between the multiple peaks.

In an embodiment, the controller 422 is configured to determine at least one of the one or more characteristics of the sensed neural activity by determining an average amplitude or a median amplitude of the multiple peaks. The controller 422 is also configured to select the one or more denervation parameters based on the one or more characteristics of the sensed neural activity by selecting at least one of the one or more denervation parameters based on the average amplitude or the median amplitude of the multiple peaks.

In an embodiment, the controller 422 is configured to determine at least one of the one or more characteristics of the sensed neural activity by fitting a curve to a portion of the signal indicative of the neural activity, and determining an area under the curve. The controller 422 is also configured to select the one or more denervation parameters based on the one or more characteristics of the sensed neural activity by selecting at least one of the one or more denervation parameters based on the area under the curve.

In an embodiment, the controller 422 is configured to determine at least one of the one or more characteristics of the sensed neural activity by determining temporal spacings between at least some of the multiple peaks relative to a cardiac cycle. The controller 422 is also configured to select the one or more denervation parameters based on the one or more characteristics of the sensed neural activity by selecting at least one of the one or more denervation parameters based on the temporal spacings between the at least some of the multiple peaks relative to the cardiac cycle.

In an embodiment, the controller 422 is further configured to diagnose a disease state based on at least one of the one or more characteristics of the sensed neural activity.

In an embodiment, the biological lumen comprises a renal artery and the nerves comprise renal nerves innervating a kidney.

In an embodiment, the sensing circuit 404 is configured to sense spontaneous neural activity of the nerves within the tissue surrounding the biological lumen using the at least one electrode 326, 327 of the catheter 302.

### EXAMPLE CROSS-SECTION OF PORTION OF SHAFT OF CATHETER

Example cross-sections of a portion of the shaft 322 is shown in FIGS. 5A and 5B. Referring to FIG. 5A, the cross-section is shown as including a main lumen 502 having a circular cross-section, and smaller lumens 504a, 504b. In order to enable the fluid to be circulated through the balloon 313, the lumen 504a is fluidically coupled to the fluidic inlet port 334a (shown in FIG. 3) to enable fluid (e.g., expelled from a pressure syringe) to be provided to and at least partially fill the balloon 313, and the lumen 504b is fluidically coupled to the fluidic outlet port 334b (shown in FIG. 3) to enable fluid to be drawn from the balloon 313 (e.g., using a vacuum syringe). FIG. 5B shows alternative cross-sections for the lumens 502, 504b, and 504c. The main lumen 502 can function as a guide wire lumen, or the main lumen can be subdivided into additional lumen, one of which can be a guide wire lumen, and another of which can be a cable lumen that is used to hold electrical cabling that is electrically coupled to a transducer (e.g., 311) or other nerve destructive means. Other variations are also possible and within the scope of the embodiments described herein.

### EXAMPLE FLUID SUPPLY SUBSYSTEM

Example details of the fluid supply subsystem 428, which were introduced above in the discussion of FIG. 4, will now be described with reference to FIG. 6. Referring to FIG. 6, the fluid supply subsystem 428 is shown as including a cartridge 630 and a reservoir 610. The reservoir 610 is shown as being implemented as a fluid bag, which can be the same or similar to an intravenous (IV) bag in that it can hang from a hook, or the like. The reservoir 610 and the cartridge 630 can be disposable and replaceable items.

The reservoir 610 is fluidically coupled to the cartridge 630 via a pair of fluidic paths, one of which is used as a fluid outlet path (that provides fluid from the reservoir to the cartridge), and the other one of which is used as a fluid inlet path (the returns fluid from the cartridge to the reservoir). The cartridge 630 is shown as including a syringe pump 640, which includes a pressure syringe 642a and a vacuum syringe 642b. The pressure syringe 642a includes a barrel 644a, a plunger 646a, and a hub 648a. Similarly, the vacuum syringe 642b includes a barrel 644b, a plunger 646b, and a hub 648b. The hub 648a, 648b of each of the syringes 642a, 642b is coupled to a respective fluid tube or hose. The cartridge 630 is also shown as including pinch valves V1, V2 and V3, pressure sensors P1, P2, and P3, and a check valve CV. While not specifically shown in FIG. 6, the syringe pump 640 can include one or more gears and step-motors, and/or the like, which are controlled by the controller 422 (in FIG. 4) to selectively maneuver the plungers 646 of the pressure syringe 642a and the vacuum syringe 642b. Alternatively, the gear(s) and/or step-motor(s) can be used to control the syringe pump 640.

In order to at least partially fill the barrel of the pressure syringe 642a with a portion of the fluid that is stored in the reservoir 610, the pinch valves V1 and V2 are closed, the pinch valve V3 is opened, and the plunger 646a of the pressure syringe 642a is pulled upon to draw fluid 613 into the barrel 644a of the of the pressure syringe 642a. The pinch valve V3 is then closed and the pinch valves V1 and V2 are opened, and then the plunger 646a of the pressure syringe 642a is pushed upon to expel fluid from the barrel 644a of the pressure syringe 642a through the fluid tube attached to the hub 648a of the pressure syringe 642a. The fluid expelled from the pressure syringe 642a enters a fluid lumen (e.g., 504a in the catheter shaft 322), via the fluidic inlet port 334a of the catheter 302, and then enters and at least partially fills the balloon 313. Simultaneously, the plunger 646b of the vacuum syringe 642b can be pulled upon to pull or draw fluid from the balloon 313 into a fluid lumen (e.g., 504b in the catheter shaft 322), through the fluidic outlet port 334b of the catheter 302, and then through fluid tube attached to the hub 648b of the vacuum syringe 642b and into the barrel 644b of the vacuum syringe 642b. In this manner, the fluid can be circulated through the balloon 313. The balloon 313 can be inflated by supplying more fluid to the balloon than is removed from the balloon. One or more of the pressure sensors P1, P2, and P3 can be used to monitor the pressure in the balloon 313 to achieve a target balloon pressure, e.g., of 70 pounds per square inch (psi), but not limited thereto. Once the balloon 313 is inflated to a target pressure, e.g., 70 psi, and/or size, the fluid can be circulated through the balloon 313 without increasing or decreasing the amount of fluid within the balloon by causing the same amount of fluid that is removed from the balloon 313 to be the same as the amount of fluid that is provided to the balloon 313. Also, once the target balloon pressure is reached, the ultrasound transducer 311 can be excited to emit ultrasound energy to treat tissue that surrounds the portion of the biological lumen (e.g., a portion of a renal artery) in which the balloon 313 and the transducer 311 are inserted. When the ultrasound transducer 311 is emitting ultrasound energy it can also be said that the ultrasound transducer 311 is performing sonication, or that sonication is occurring. During the sonication, cooling fluid should be circulated through the balloon 313 by continuing to push on the plunger 646a of the pressure syringe 642a and continuing to pull on the plunger 646b of the vacuum syringe 642b.

As was explained above in the discussion of FIG. 1, in accordance with certain embodiments of the present technology, the flow rate of the cooling fluid that is circulated through the balloon 313 is one example of a denervation parameter that can be selected and used (e.g., by the controller 422) to perform a denervation parameter. Still referring to FIG. 6, the fluid supply subsystem 428 can include a cooling coil 650 within or adjacent to the reservoir 610 to control the temperature of the colling fluid 613. As was explained above in the discussion of FIG. 1, in accordance with certain embodiments of the present technology, the temperature of the cooling fluid that is circulated through the balloon is another example of a denervation parameter that can be selected and used (e.g., by the controller 422) to perform a denervation procedure. The controller 422 introduced in FIG. 4 can be used to control the flow rate of the cooling fluid that is circulated through the balloon 313 by controlling the syringe pump 640. Alternatively, or additionally, the controller 422 can control the temperature of the cooling fluid 613 by controlling the temperature of the cooling coil 650.

After the sonication is completed, and the balloon 313 is to be deflated so that the catheter 302 can be removed from the biological lumen, the cooling fluid should be returned from the barrel 644b of the vacuum syringe 642b to the reservoir 610. In order to return the cooling fluid from the barrel 644b of the vacuum syringe 642b to the reservoir 610, the pinch valves V1, V2, and V3 are all closed, and the plunger of the vacuum syringe 642b is pushed on to expel the cooling fluid out of the barrel of the vacuum syringe 642b, past the check valve CV, and into the reservoir 610.

The pressure sensors P1, P2, and P3 can be used to monitor the fluidic pressure at various points along the various fluidic paths within the cartridge 630, which pressure measurements can be provided to the controller 422 as feedback that is used for controlling the syringe pump 640 and/or for other purposes, such as, but not limited to, determining the fluidic pressure within the balloon 313. Additionally, flow rate sensors F1 and F2 can be used, respectively, to monitor the flow rate of the fluid that is being injected (aka pushed, provided, or supplied) into the balloon 313, and to monitor the flow rate of the fluid that is being drawn (aka pulled or removed) from the balloon 313. The pressure measurements obtained from the pressure sensors P1, P2, and P3 can be provided to the controller 422 so that the controller 422 can monitor the balloon pressure. Additionally, flow rate measurements obtained from the flow rate sensors F1 and F2 can be provided to the controller 422 so that the controller 422 can monitor the flow rate of fluid being pushed into and pulled from the balloon 313. It would also be possible for one or more pressure sensors and/or flow rate sensors to be located at additional or alternative locations along the fluidic paths that provide fluid to and from the balloon 313.

### EXAMPLE TRANSDUCER

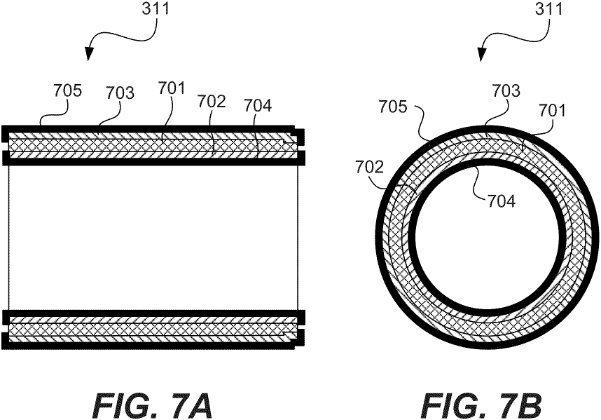

FIGS. 7A and 7B illustrate, respectively, a longitudinal cross-sectional view and a radial cross-sectional view of an example transducer 311 of the catheter 302 introduced above in the discussion of FIGS. 3A and 3B. The transducer 311, which in the embodiment show in FIGS. 7A and 7B is an ultrasound transducer, includes a piezoelectric transducer body 701 that comprises a hollow tube of piezoelectric material having an inner surface and an outer surface, with an inner electrode 702 disposed on the inner surface of the hollow tube of piezoelectric material, and an outer electrode 703 disposed on the outer surface of the hollow tube of piezoelectric material. The hollow tube of piezoelectric material, or more generally the piezoelectric transducer body 701, is cylindrically shaped and has a circular radial cross-section. However, in alternative embodiments the transducer body 701 can have other shapes besides being cylindrical with a circular radial cross-section. The inner electrode 702 is covered by an electrical insulator 704, and the outer electrode 703 is covered by an electrical insulator 705. It is also possible that only one of the electrodes 702, 703 is covered by an electrical insulator. Other variations are also possible and within the scope of the embodiments described herein.

### EXAMPLE SYSTEMS AND METHODS

Example 1. A tissue treatment system, comprising: a signal generator; a sensing circuit coupleable to at least one electrode of a catheter that is insertable into a biological lumen, the sensing circuit configured to sense neural activity of nerves within tissue surrounding the biological lumen using the at least one electrode of the catheter while the catheter is inserted into the biological lumen; and a controller communicatively coupled to the signal generator and the sensing circuit; the controller comprising one or more processors and configured to: determine one or more characteristics of the sensed neural activity of the nerves within the tissue surrounding the biological lumen, the one or more characteristics indicative of one or more of a size, type, function or health of the nerves and/or indicative of proximity of the nerves relative to the at least one electrode of the catheter; select one or more denervation parameters based on the one or more characteristics of the sensed neural activity; and control the signal generator to generate, using the selected one or more denervation parameters, signals for performing a denervation procedure intended to denervate at least some of the nerves for which the neural activity was sensed.

Example 2. The system of example 1, further comprising the catheter, wherein: the sensing circuit is configured to sense the neural activity of the nerves using the at least one electrode; and the signal generator is configured to generate the signals for performing the denervation procedure.

Example 3. The system of example 1 or 2, wherein: the sensing circuit is configured to sense evoked neural activity of the nerves within the tissue surrounding the biological lumen using the at least one electrode of the catheter; and the evoked neural activity is responsive to electrical stimulation delivered using at least one other electrode of the catheter.

Example 4. The system of example 1, further comprising: a first catheter comprising the at least one electrode; and a second catheter comprising at least one electrode configured to emit radio frequency (RF) energy and/or an ultrasound transducer configured to emit ultrasound energy; wherein the sensing circuit is configured to sense the neural activity of the nerves using the at least one electrode of the first catheter; and wherein the signal generator is configured to generate the signals for performing the denervation procedure using the at least one electrode of the second catheter, or the ultrasound transducer of the second catheter.

Example 5. The system of any one of examples 1 through 4, wherein the one or more denervation parameters selected by the controller comprise one or more of amplitude, power, duration, frequency, and duty cycle of ultrasound energy emitted by an ultrasound transducer.

Example 6. The system of example 5, wherein the ultrasound transducer is located within a balloon that is at least partially filled with a cooling fluid that is circulated through the balloon in order to cool at least a portion of the tissue surrounding the biological lumen proximate the balloon, and wherein the one or more denervation parameters selected by the controller also comprise at least one of a flow rate or a temperature associated with the cooling fluid.

Example 7. The system of any one of examples 1 through 4, wherein the denervation parameters selected by the controller comprise one or more of amplitude, power, duration, frequency, and duty cycle of radio frequency (RF) energy emitted by at least one electrode.

Example 8. The system of any one of examples 1 through 7, wherein the controller is configured to: determine at least one of the one or more characteristics of the sensed neural activity by determining a minimal amount of stimulation energy needed to evoke a neural response by the nerves within tissue surrounding the biological lumen; and select the one or more denervation parameters based on the one or more characteristics of the sensed neural activity by selecting at least one of the one or more denervation parameters based on the minimal amount of stimulation energy needed to evoke the neural response by the nerves within tissue surrounding the biological lumen.

Example 9. The system of any one of examples 1 through 8, wherein the controller is configured to: determine at least one of the one or more characteristics of the sensed neural activity by determining an amount of stimulation energy needed to cause saturation of an evoked neural response of the nerves within tissue surrounding the biological lumen; and select the one or more denervation parameters based on the one or more characteristics of the sensed neural activity by selecting at least one of the one or more denervation parameters based on the amount of stimulation energy needed to cause saturation of the evoked neural response of the nerves within tissue surrounding the biological lumen.

Example 10. The system of any one of examples 1 through 9, wherein the controller is configured to select at least one of the one or more denervation parameters using one or more tables accessed by the controller from a memory.

Example 11. The system of any one of examples 1 through 9, wherein the controller is configured to select at least one of the one or more denervation parameters using a machine learning model implemented by at least one of the one or more processors of the controller.

Example 12. The system of any one of examples 1 through 11, wherein: the sensing circuit is configured to sense the neural activity of the nerves within the tissue surrounding the biological lumen by sensing a signal indicative of the neural activity, the signal indicative of the neural activity including multiple peaks temporally spaced apart from one another; and the controller is configured to determine at least one of the one or more characteristics of the sensed neural activity based on at least one of amplitudes of the multiple peaks or based on temporal spacings between the multiple peaks.

Example 13. The system of example 12, wherein the controller is configured to: determine at least one of the one or more characteristics of the sensed neural activity by determining an average amplitude or a median amplitude of the multiple peaks; and select the one or more denervation parameters based on the one or more characteristics of the sensed neural activity by selecting at least one of the one or more denervation parameters based on the average amplitude or the median amplitude of the multiple peaks.

Example 14. The system of any one of examples 12 or 13, wherein the controller is configured to: determine at least one of the one or more characteristics of the sensed neural activity by fitting a curve to a portion of the signal indicative of the neural activity, and determining an area under the curve; and select the one or more denervation parameters based on the one or more characteristics of the sensed neural activity by selecting at least one of the one or more denervation parameters based on the area under the curve.

Example 15. The system of any one of examples 12 through 14, wherein the controller is configured to: determine at least one of the one or more characteristics of the sensed neural activity by determining temporal spacings between at least some of the multiple peaks relative to a cardiac cycle; and select the one or more denervation parameters based on the one or more characteristics of the sensed neural activity by selecting at least one of the one or more denervation parameters based on the temporal spacings between the at least some of the multiple peaks relative to the cardiac cycle.

Example 16. The system of any one of examples 1 through 15, wherein the controller is further configured to diagnose a disease state based on at least one of the one or more characteristics of the sensed neural activity.

Example 17. The system of any one of examples 1 through 15, wherein the biological lumen comprises a renal artery and the nerves comprise renal nerves innervating a kidney.

Example 18. The system of any one of examples 1 through 15, wherein the sensing circuit is configured to sense spontaneous neural activity of the nerves within the tissue surrounding the biological lumen using the at least one electrode of the catheter.

Example 19. A tissue treatment method, comprising: sensing neural activity of nerves within tissue surrounding a biological lumen; determining one or more characteristics of the sensed neural activity of the nerves within the tissue surround the biological lumen, the one or more characteristics indicative of one or more of a size, type, function, or health of the nerves, and/or proximity of the nerves relative to at least one electrode of a catheter inserted in the biological lumen; selecting one or more denervation parameters based on the one or more characteristics of the sensed neural activity, the one or more denervation parameters for use in performing a denervation procedure intended to denervate at least some of the nerves for which the neural activity was sensed, the selecting one or more denervation parameters performed using one or more processors; and performing the denervation procedure using the one or more denervation parameters selected to thereby denervate at least some of the nerves for which the neural activity was sensed.

Example 20. The method of example 19, wherein the biological lumen comprises a renal artery and the nerves comprise renal nerves innervating a kidney.

Example 21. The method of any one of examples 19 or 20, wherein the neural activity that is sensed comprises spontaneous neural activity.

Example 22. The method of any one of examples 19 or 20, wherein the neural activity that is sensed comprises evoked neural activity that is responsive to electrical stimulation delivered using the same catheter used to sense the evoked neural activity.

Example 23. The method of any one of example 19, 20 or 22, wherein: the determining the one or more characteristics of the sensed neural activity comprises determining a minimal amount of stimulation energy needed to evoke a neural response by the nerves within tissue surrounding the biological lumen; and the selecting the one or more denervation parameters based on the one or more characteristics of the sensed neural activity comprises selecting at least one of the one or more denervation parameters based on the minimal amount of stimulation energy needed to evoke the neural response by the nerves within tissue surrounding the biological lumen.

Example 24. The method of any one of examples 19, 20 or 22, wherein: the determining the one or more characteristics of the sensed neural activity comprises determining an amount of stimulation energy needed to cause saturation of an evoked neural response of the nerves within tissue surrounding the biological lumen; and the selecting the one or more denervation parameters based on the one or more characteristics of the sensed neural activity comprises selecting at least one of the one or more denervation parameters based on the amount of stimulation energy needed to cause saturation of the evoked neural response of the nerves within tissue surrounding the biological lumen.

Example 25. The method of any one of examples 19 through 24, wherein the performing the denervation procedure comprising using an ultrasound transducer inserted into the biological lumen to emit ultrasound energy, and wherein the one or more denervation parameters selected comprise one or more of amplitude, power, duration, frequency, and duty cycle of the ultrasound energy.

Example 26. The method of example 25, wherein the ultrasound transducer is located within a balloon that is at least partially filled with a cooling fluid that is circulated through the balloon in order to cool at least a portion of the tissue surrounding the biological lumen proximate the balloon, and wherein the one or more denervation parameters selected also specify at least one of a flow rate or a temperature associated with the cooling fluid.

Example 27. The method of any one of examples 19 through 24, wherein the performing the denervation procedure comprising using one or more electrodes inserted into the biological lumen to emit RF energy, and wherein the one or more denervation parameters selected comprise one or more of amplitude, power, duration, frequency, and duty cycle of the RF energy.

Example 28. The method of any one of examples 19 through 27, wherein the selecting the one or more denervation parameters is performed using one or more tables accessed by at least one of the one or more processors.

Example 29. The method of any one of examples 19 through 27, wherein the selecting the one or more denervation parameters is performed using a machine learning model implemented by at least one of the one or more processors.

Example 30. The method of any one of examples 19 through 29, wherein: the sensing neural activity of the nerves within the tissue surrounding the biological lumen comprises sensing a signal indicative of the neural activity, the signal indicative of the neural activity including multiple peaks temporally spaced apart from one another; and the determining the one or more characteristics of the sensed neural activity comprises determining at least one of the one or more characteristics based on at least one of amplitudes of the multiple peaks or temporal spacings between the multiple peaks.

Example 31. The method of example 30, wherein: the determining the one or more characteristics of the sensed neural activity comprises determining an average amplitude or a median amplitude of the multiple peaks; and the selecting the one or more denervation parameters based on the one or more characteristics of the sensed neural activity comprises selecting at least one of the one or more denervation parameters based on the average amplitude or the median amplitude of the multiple peaks.

Example 32. The method of any one of examples 30 or 31, wherein: the determining the one or more characteristics of the sensed neural activity comprises fitting a curve to a portion of the signal indicative of the neural activity, and determining an area under the curve; and the selecting the one or more denervation parameters based on the one or more characteristics of the sensed neural activity comprises selecting at least one of the one or more denervation parameters based on the area under the curve.

Example 33. The method of any one of examples 30 through 32, wherein: the determining the one or more characteristics of the sensed neural activity comprises determining temporal spacings between at least some of the multiple peaks relative to a cardiac cycle; and the selecting the one or more denervation parameters based on the one or more characteristics of the sensed neural activity comprises selecting at least one of the one or more denervation parameters based on the temporal spacings between the at least some of the multiple peaks relative to the cardiac cycle.

Example 34. The method of any one of examples 19 through 33, further comprising: diagnosing a disease state based on at least one of the one or more characteristics of the sensed neural activity.

Example 35. The method of any one of examples 19 through 34, wherein: the sensing is performed using at least one electrode of a catheter inserted into the biological lumen; and the denervation procedure is performed using the same catheter used to sense the neural activity, or using a separate catheter inserted into the biological lumen after the catheter used to sense the neural activity has been removed.

Although several embodiments and examples are disclosed herein, the present application extends beyond the specifically disclosed embodiments to other alternative embodiments and/or uses of the inventions and modifications and equivalents thereof. It is also contemplated that various combinations or subcombinations of the specific features and aspects of the embodiments may be made and still fall within the scope of the inventions. Accordingly, it should be understood that various features and aspects of the disclosed embodiments can be combine with or substituted for one another in order to form varying modes of the disclosed inventions. Thus, it is intended that the scope of the present inventions herein disclosed should not be limited by the particular disclosed embodiments described above, but should be determined only by a fair reading of the claims that follow.

While the inventions are susceptible to various modifications, and alternative forms, specific examples thereof have been shown in the drawings and are herein described in detail. It should be understood, however, that the inventions are not to be limited to the particular forms or methods disclosed, but, to the contrary, the inventions are to cover all modifications, equivalents, and alternatives falling within the spirit and scope of the various embodiments described and the appended claims. Any methods disclosed herein need not be performed in the order recited.

What is claimed is:

1. A tissue treatment system, comprising:
a signal generator;
a sensing circuit coupleable to at least one electrode of a catheter that is insertable into a biological lumen, the sensing circuit configured to sense neural activity of nerves within tissue surrounding the biological lumen using the at least one electrode of the catheter while the catheter is inserted into the biological lumen; and
a controller communicatively coupled to the signal generator and the sensing circuit;
the controller comprising one or more processors and configured to:
determine one or more characteristics of the sensed neural activity of the nerves within the tissue surrounding the biological lumen, the one or more characteristics indicative of one or more of a size, type, function or health of the nerves and/or indicative of proximity of the nerves relative to the at least one electrode of the catheter;
select one or more denervation parameters based on the one or more characteristics of the sensed neural activity; and
control the signal generator to generate, using the selected one or more denervation parameters, signals for performing a denervation procedure intended to denervate at least some of the nerves for which the neural activity was sensed.

2. The system of claim 1, further comprising the catheter, wherein:
the sensing circuit is configured to sense the neural activity of the nerves using the at least one electrode; and
the signal generator is configured to generate the signals for performing the denervation procedure.

3. The system of claim 2, wherein:
the sensing circuit is configured to sense evoked neural activity of the nerves within the tissue surrounding the biological lumen using the at least one electrode of the catheter; and
the evoked neural activity is responsive to electrical stimulation delivered using at least one other electrode of the catheter.

4. The system of claim 1, further comprising:
a first catheter comprising the at least one electrode; and
a second catheter comprising at least one electrode configured to emit radio frequency (RF) energy and/or an ultrasound transducer configured to emit ultrasound energy;
wherein the sensing circuit is configured to sense the neural activity of the nerves using the at least one electrode of the first catheter; and
wherein the signal generator is configured to generate the signals for performing the denervation procedure using the at least one electrode of the second catheter, or the ultrasound transducer of the second catheter.

5. The system of claim 1, wherein the one or more denervation parameters selected by the controller comprise one or more of amplitude, power, duration, frequency, and duty cycle of ultrasound energy emitted by an ultrasound transducer.

6. The system of claim 5, wherein the ultrasound transducer is located within a balloon that is at least partially filled with a cooling fluid that is circulated through the balloon in order to cool at least a portion of the tissue surrounding the biological lumen proximate the balloon, and wherein the one or more denervation parameters selected by the controller also comprise at least one of a flow rate or a temperature associated with the cooling fluid.

7. The system of claim 1, wherein the denervation parameters selected by the controller comprise one or more of amplitude, power, duration, frequency, and duty cycle of radio frequency (RF) energy emitted by at least one electrode.

8. The system of claim 1, wherein the controller is configured to:
determine at least one of the one or more characteristics of the sensed neural activity by determining a minimal amount of stimulation energy needed to evoke a neural response by the nerves within tissue surrounding the biological lumen; and
select the one or more denervation parameters based on the one or more characteristics of the sensed neural activity by selecting at least one of the one or more denervation parameters based on the minimal amount of stimulation energy needed to evoke the neural response by the nerves within tissue surrounding the biological lumen.

9. The system of claim 1, wherein the controller is configured to:
determine at least one of the one or more characteristics of the sensed neural activity by determining an amount of stimulation energy needed to cause saturation of an evoked neural response of the nerves within tissue surrounding the biological lumen; and
select the one or more denervation parameters based on the one or more characteristics of the sensed neural activity by selecting at least one of the one or more denervation parameters based on the amount of stimulation energy needed to cause saturation of the evoked neural response of the nerves within tissue surrounding the biological lumen.

10. The system of claim 1, wherein the controller is configured to select at least one of the one or more denervation parameters using one or more tables accessed by the controller from a memory.

11. The system of claim 1, wherein the controller is configured to select at least one of the one or more denervation parameters using a machine learning model implemented by at least one of the one or more processors of the controller.

12. The system of claim 1, wherein:
the sensing circuit is configured to sense the neural activity of the nerves within the tissue surrounding the biological lumen by sensing a signal indicative of the neural activity, the signal indicative of the neural activity including multiple peaks temporally spaced apart from one another; and
the controller is configured to determine at least one of the one or more characteristics of the sensed neural activity based on at least one of amplitudes of the multiple peaks or based on temporal spacings between the multiple peaks.

13. The system of claim 12, wherein the controller is configured to:
determine at least one of the one or more characteristics of the sensed neural activity by determining an average amplitude or a median amplitude of the multiple peaks; and
select the one or more denervation parameters based on the one or more characteristics of the sensed neural activity by selecting at least one of the one or more denervation parameters based on the average amplitude or the median amplitude of the multiple peaks.

14. The system of claim 12, wherein the controller is configured to:
determine at least one of the one or more characteristics of the sensed neural activity by fitting a curve to a portion of the signal indicative of the neural activity, and determining an area under the curve; and
select the one or more denervation parameters based on the one or more characteristics of the sensed neural activity by selecting at least one of the one or more denervation parameters based on the area under the curve.

15. The system of claim 12, wherein the controller is configured to:
determine at least one of the one or more characteristics of the sensed neural activity by determining temporal spacings between at least some of the multiple peaks relative to a cardiac cycle; and
select the one or more denervation parameters based on the one or more characteristics of the sensed neural activity by selecting at least one of the one or more denervation parameters based on the temporal spacings between the at least some of the multiple peaks relative to the cardiac cycle.

16. The system of claim 1, wherein the controller is further configured to diagnose a disease state based on at least one of the one or more characteristics of the sensed neural activity.

17. The system of claim 1, wherein the biological lumen comprises a renal artery and the nerves comprise renal nerves innervating a kidney.

18. The system of claim 1, wherein the sensing circuit is configured to sense spontaneous neural activity of the nerves within the tissue surrounding the biological lumen using the at least one electrode of the catheter.

19. A tissue treatment method, comprising:
sensing neural activity of nerves within tissue surrounding a biological lumen;
determining one or more characteristics of the sensed neural activity of the nerves within the tissue surround the biological lumen, the one or more characteristics indicative of one or more of a size, type, function, or health of the nerves, and/or proximity of the nerves relative to at least one electrode of a catheter inserted in the biological lumen;
selecting one or more denervation parameters based on the one or more characteristics of the sensed neural activity, the one or more denervation parameters for use in performing a denervation procedure intended to denervate at least some of the nerves for which the neural activity was sensed, the selecting one or more denervation parameters performed using one or more processors; and
performing the denervation procedure using the one or more denervation parameters selected to thereby denervate at least some of the nerves for which the neural activity was sensed.

20. The method of claim 19, wherein the biological lumen comprises a renal artery and the nerves comprise renal nerves innervating a kidney.

21. The method of claim 19, wherein the neural activity that is sensed comprises spontaneous neural activity.

22. The method of claim 19, wherein the neural activity that is sensed comprises evoked neural activity that is responsive to electrical stimulation delivered using the same catheter used to sense the evoked neural activity.

23. The method of claim 19, wherein:
the determining the one or more characteristics of the sensed neural activity comprises determining a minimal amount of stimulation energy needed to evoke a neural response by the nerves within tissue surrounding the biological lumen; and
the selecting the one or more denervation parameters based on the one or more characteristics of the sensed neural activity comprises selecting at least one of the one or more denervation parameters based on the minimal amount of stimulation energy needed to evoke the neural response by the nerves within tissue surrounding the biological lumen.

24. The method of claim 19, wherein:
the determining the one or more characteristics of the sensed neural activity comprises determining an amount of stimulation energy needed to cause saturation of an evoked neural response of the nerves within tissue surrounding the biological lumen; and
the selecting the one or more denervation parameters based on the one or more characteristics of the sensed neural activity comprises selecting at least one of the one or more denervation parameters based on the amount of stimulation energy needed to cause saturation of the evoked neural response of the nerves within tissue surrounding the biological lumen.

25. The method of claim 19, wherein the performing the denervation procedure comprising using an ultrasound transducer inserted into the biological lumen to emit ultrasound energy, and wherein the one or more denervation parameters selected comprise one or more of amplitude, power, duration, frequency, and duty cycle of the ultrasound energy.

26. The method of claim 25, wherein the ultrasound transducer is located within a balloon that is at least partially filled with a cooling fluid that is circulated through the balloon in order to cool at least a portion of the tissue surrounding the biological lumen proximate the balloon, and wherein the one or more denervation parameters selected also specify at least one of a flow rate or a temperature associated with the cooling fluid.

27. The method of claim 19, wherein the performing the denervation procedure comprising using one or more electrodes inserted into the biological lumen to emit RF energy, and wherein the one or more denervation parameters selected comprise one or more of amplitude, power, duration, frequency, and duty cycle of the RF energy.

28. The method of claim 19, wherein the selecting the one or more denervation parameters is performed using one or more tables accessed by at least one of the one or more processors.

29. The method of claim 19, wherein the selecting the one or more denervation parameters is performed using a machine learning model implemented by at least one of the one or more processors.

30. The method of claim 19, wherein:
the sensing neural activity of the nerves within the tissue surrounding the biological lumen comprises sensing a signal indicative of the neural activity, the signal indicative of the neural activity including multiple peaks temporally spaced apart from one another; and
the determining the one or more characteristics of the sensed neural activity comprises determining at least one of the one or more characteristics based on at least one of amplitudes of the multiple peaks or temporal spacings between the multiple peaks.

31. The method of claim 30, wherein:
the determining the one or more characteristics of the sensed neural activity comprises determining an average amplitude or a median amplitude of the multiple peaks; and
the selecting the one or more denervation parameters based on the one or more characteristics of the sensed neural activity comprises selecting at least one of the one or more denervation parameters based on the average amplitude or the median amplitude of the multiple peaks.

32. The method of claim 31, wherein:
the determining the one or more characteristics of the sensed neural activity comprises fitting a curve to a portion of the signal indicative of the neural activity, and determining an area under the curve; and
the selecting the one or more denervation parameters based on the one or more characteristics of the sensed neural activity comprises selecting at least one of the one or more denervation parameters based on the area under the curve.

33. The method of claim 30, wherein:
the determining the one or more characteristics of the sensed neural activity comprises determining temporal spacings between at least some of the multiple peaks relative to a cardiac cycle; and
the selecting the one or more denervation parameters based on the one or more characteristics of the sensed neural activity comprises selecting at least one of the one or more denervation parameters based on the temporal spacings between the at least some of the multiple peaks relative to the cardiac cycle.

34. The method of claim 19, further comprising:
diagnosing a disease state based on at least one of the one or more characteristics of the sensed neural activity.

35. The method of claim 19, wherein:
the sensing is performed using at least one electrode of a catheter inserted into the biological lumen; and
the denervation procedure is performed using the same catheter used to sense the neural activity, or using a separate catheter inserted into the biological lumen after the catheter used to sense the neural activity has been removed.
